(19) United States
(12) Patent Application Publication
(10) Pub. No.: US 2012/0329787 A1
(43) Pub. Date: Dec. 27, 2012

(54) FUNGICIDAL PYRANONES AND OXAZINONES

(75) Inventor: THOMAS MARTIN STEVENSON, Newark, DE (US)

(73) Assignee: E.I. DU PONT DE NEMOURS AND COMPANY, Wilmington, DE (US)

(21) Appl. No.: 13/166,248

(22) Filed: Jun. 22, 2011

(51) Int. Cl. A01N 43/72; C07D 309/38; A01N 43/78; A01P 3/00; C07D 417/04; C07D 265/02; A01N 43/16

(52) U.S. Cl. 514/228.8; 544/63; 549/416; 514/460; 548/204; 514/365

(57) ABSTRACT

Disclosed are compounds of Formula 1, including all geometric and stereoisomers, tautomers, N-oxides, and salts thereof,

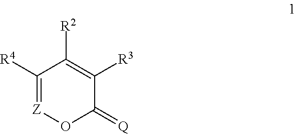

wherein
Q, Z, R², R³ and R⁴ are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain pyranones and oxazinones, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

Kuninobu et al., Chemical Communications 2008, 47, 6360-6362 discloses certain pyranones derivatives; and Fusco et al., Gazzetta Chimica Italiana 1937, 67, 248-256 discloses certain oxazinones.

### SUMMARY OF THE INVENTION

This invention is directed to compounds of Formula 1 (including all stereoisomers), N-oxides, and salts thereof, agricultural compositions containing them and their use as fungicides:

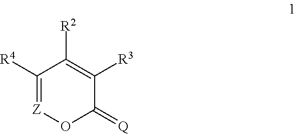

wherein
Q is O or S;
Z is N or CR¹;
R¹ is H, halogen, cyano, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ cyanoalkyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylamino or C₂-C₄ dialkylamino;
R² is a phenyl ring substituted with up to 5 substituents independently selected from R⁵; or a 3- to 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from R⁵ on carbon atom ring members and R^{5a} on nitrogen atom ring members;
R³ is a phenyl ring optionally substituted with up to 5 substituents independently selected from R⁶; or a 3- to 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from R⁶ on carbon atom ring members and R^{6a} on nitrogen atom ring members; or H, halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₃-C₆ cycloalkylamino, C₂-C₆ alkylcarbonylamino, C₂-C₆ haloalkylcarbonylamino, C₁-C₆ alkylsulfonylamino or C₁-C₆ haloalkylsulfonylamino;
R⁴ is a phenyl ring optionally substituted with up to 5 substituents independently selected from R⁷; or a 3- to 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from R⁷ on carbon atom ring members and R^{7a} on nitrogen atom ring members; or halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₃-C₆ cycloalkylamino, C₂-C₆ alkylcarbonylamino, C₂-C₆ haloalkylcarbonylamino, C₁-C₆ alkylsulfonylamino or C₁-C₆ haloalkylsulfonylamino;
each R⁵ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino; or
a pair of R⁵ attached to the same carbon atom are taken together to form a 5- to 7-membered spirocyclic ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro, C₁-C₂ alkyl and C₁-C₂ alkoxy on carbon atom ring members and cyano, C₁-C₂ alkyl and C₁-C₂ alkoxy on nitrogen atom ring members;
each R^{5a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl; or
a pair of R⁵ and R^{5a} attached to adjacent ring atoms are taken together to form a 5- to 7-membered ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl, and C₁-C₂ alkoxy on nitrogen atom ring members;
each R⁶ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino; or
a pair of R⁶ attached to the same carbon atom are taken together to form a 5- to 7-membered spirocyclic ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl and C₁-C₂ alkoxy on nitrogen atom ring members;
each R^{6a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl; or
a pair of R⁶ and R^{6a} attached to adjacent ring atoms are taken together to form a 5- to 7-membered ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl and C₁-C₂ alkoxy on nitrogen atom ring members;
each R⁷ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino; or
a pair of R⁷ attached to adjacent carbon atoms are taken together to form a 5- to 7-membered ring containing ring members selected from carbon atoms and optionally up to 4 heteroatoms selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl and C₁-C₂ alkoxy on nitrogen atom ring members;
each R^{7a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl;
each R⁸ is independently H or C₁-C₃ alkyl; and p and q are independently 0, 1 or 2 in each instance of S(═O)ₚ(═NR⁸)_{q}, provided that the sum of p and q is 0, 1 or 2;
provided that the compound is other than
4-(4-chlorophenyl)-3,5-dimethyl-6H-1,2-oxazin-6-one,
4-[4-(dimethylamino)phenyl]-3-phenyl-6H-1,2-oxazin-6-one,
3,5-dimethyl-4-(3-pyridinyl)-6H-1,2-oxazin-6-one,
4-(4-methoxyphenyl)-3-phenyl-6H-1,2-oxazin-6-one,
3,4-diphenyl-6H-1,2-oxazin-6-one,
4,5-diphenyl-2H-pyran-2-one, or
5,6-dimethyl-3,4-diphenyl-2H-pyran-2-one.

More particularly, this invention pertains to a compound of Formula 1 (including all stereoisomers), an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a fungicidal composition comprising (a) at least one compound of the invention (i.e. in a fungicidally effective amount); and (b) at least one additional compound of the invention (i.e. in a fungicidally effective amount) which is a regisomer of component (a) (e.g., at least one compound of Formula 1 which is a regiosmer of at least one compound contained in component (a)).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

In the above recitations, the term “alkyl”, used either alone or in compound words such as “alkylthio” or “haloalkyl” includes straight-chain and branched alkyl, such as, methyl, ethyl, n-propyl, i-propyl, and the different butyl, pentyl and hexyl isomers. “Alkenyl” includes straight-chain and branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain and branched alkynes such as ethynyl, 1-propynyl, 2-propynyl, and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, i-propyloxy, and the different butoxy, pentoxy and hexyloxy isomers. The term “alkylthio” includes straight-chain and branched alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O), and the different butylsulfinyl, pentylsulfinyl and hexylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl and hexylsulfonyl isomers. “Alkylamino” includes an NH radical substituted with a straight-chain or branched alkyl group. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃C(═O), CH₃CH₂OC(═O), CH₃CH₂CH₂C(═O), (CH₃)₂CHOC(═O), and the different butoxy- and pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O), and the different butylamino- and pentylaminocarbonyl isomers. Examples of “dialkylaminocarbonyl” include (CH₃)₂NC(═O), (CH₃CH₂)₂NC(═O), CH₃CH₂(CH₃)NC(═O), (CH₃)₂CH(CH₃)NC(═O) and CH₃CH₂CH₂(CH₃)NC(═O).

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on another alkoxy moiety. Examples of “alkoxyalkoxy” include CH₃OCH₂O, CH₃CH₂OCH₂O and (CH₃)₂CHOCH₂O.

“Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂; “alkylsulfinylalkyl” and “alkylsulfonylalkyl” include the corresponding sulfoxides and sulfones, respectively.

“Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂. Examples of “dialkylaminoalkyl” include (CH₃)₂NCH₂ and CH₃CH₂(CH₃)NCH₂CH₂.

“Alkylaminoalkoxy” denotes alkylamino substitution on alkoxy. Examples of “alkylaminoalkoxy” include CH₃NHCH₂CH₂O, CH₃NHCH₂CH₂CH₂O and CH₃CH(CH₃)NHCH₂CH₂O.

“Alkylcarbonylthio” denotes a straight-chain or branched alkylcarbonyl attached to and linked through a sulfur atom. Examples of “alkylcarbonylthio” include CH₃C(═O)S, CH₃CH₂CH₂C(═O)S and (CH₃)₂CHC(═O)S. The term “alkylcarbonylamino” denotes alkylcarbonyl attached to and linked through an NH radical. Examples of “alkylcarbonylamino” include CH₃CH₂C(═O)NH and CH₃CH₂CH₂C(═O)NH. The term “alkylcarbonyloxy” denotes a straight-chain or branched alkylcarbonyl bonded and linked through an oxygen atom. Examples of “alkylcarbonyloxy” include CH₃CH₂C(═O)O and (CH₃)₂CHC(═O)O.

“Alkylsulfonylamino” denotes an NH radical substituted with alkylsulfonyl. Examples of “alkylsulfonylamino” include CH₃CH₂S(═O)₂NH and (CH₃)₂CHS(═O)₂NH.

“Alkoxycarbonylalkyl” denotes alkoxycarbonyl substitution on straight-chain or branched alkyl. Examples of “alkoxycarbonylalkyl” include CH₃C(═O)CH₂CH(CH₃), CH₃CH₂C(═O)CH₂CH₂ and (CH₃)₂CHOC(═O)CH₂.

“Cyanoalkyl” denotes an alkyl group substituted with one cyano group. Examples of “cyanoalkyl” include NCCH₂, NCCH₂CH₂ and CH₃CH(CN)CH₂.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to a straight-chain or branched alkyl group. The term “alkylcycloalkyl” denotes alkyl substitution on a cycloalkyl moiety and includes, for example, ethylcyclopropyl, i-propylcyclobutyl, methylcyclopentyl and methylcyclohexyl. “Cycloalkenyl” includes groups such as cyclopentenyl and cyclohexenyl as well as groups with more than one double bond such as 1,3- or 1,4-cyclohexadienyl. The term “cycloalkoxy” denotes cycloalkyl attached to and linked through an oxygen atom such as cyclopentyloxy and cyclohexyloxy.

“Alkylcycloalkylalkyl” denotes an alkyl group substituted with alkylcycloalkyl. Examples of “alkylcycloalkylalkyl” include methylcyclohexylmethyl and ethyl-cyclopropylmethyl. The term “cycloalkylcycloalkyl” denotes cycloalkyl substitution on another cycloalkyl ring, wherein each cycloalkyl ring independently has from 3 to 6 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

“Cycloalkylamino” denotes an NH radical substituted with cycloalkyl. Examples of “cycloalkylamino” include cyclopropylamino and cyclohexylamino.

“Cycloalkylcarbonyl” denotes cycloalkyl bonded to and linked through a C(═O) group including, for example, cyclopropylcarbonyl and cyclopentylcarbonyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkenyl”, “haloalkynyl” “haloalkoxy”, “haloalkylthio”, “haloalkylamino”, “haloalkylcarbonyl”, “haloalkylcarbonylamino” “haloalkylsulfinyl”, “haloalkylsulfonyl”, “halocycloalkyl”, “halodialkylamino”, and the like, are defined analogously to the term “haloalkyl”. Examples of “haloalkenyl” include Cl₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkylamino” include CF₃(CH₃)CHNH, (CF₃)₂CHNH and CH₂ClCH₂NH. Examples of “haloalkylcarbonyl” include CH₂ClCH₂C(O), CH₂ClCH₂CH₂C(O) and CH₃CH₂CHBrCH₂C(O). Examples of “haloalkylcarbonylamino” include CHClCH₂C(O)NH, CH₃CHBrCH₂CH₂C(O)NH and (CH₂Cl)(CH₃)CHC(O)NH. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S (═O)₂. Examples of “halocycloalkyl” include 2-chlorocyclopropyl, 2-fluorocyclobutyl, 3-bromocyclopentyl and 4-chorocyclohexyl. The term “halodialkyl”, either alone or in compound words such as “halodialkylamino”, means at least one of the two alkyl groups is substituted with at least one halogen atom, and independently each alkyl group may be partially or fully substituted with halogen atoms which may be the same or different. Examples of “halodialkylamino” include (BrCH₂CH₂)₂N and BrCH₂CH₂(ClCH₂CH₂)N.

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

“Trialkylsilyl” includes 3 branched and/or straight-chain alkyl radicals attached to and linked through a silicon atom, such as trimethylsilyl, triethylsilyl and tert-butyldimethylsilyl.

The total number of carbon atoms in a substituent group is indicated by the “Cᵢ-Cⱼ” prefix where i and j are numbers from 1 to 12. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring or ring system means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 3 substituents independently selected from R⁵ on carbon atom ring members” means that 0, 1, 2 or 3 substituents can be present (if the number of potential connection points allows). Similarly, the phrase “optionally substituted with up to 5 substituents independently selected from R⁵ on carbon atom ring members” means that 0, 1, 2, 3, 4 or 5 substituents can be present if the number of available connection points allows. When a range specified for the number of substituents (e.g., r being an integer from 0 to 5 in Exhibit 1) exceeds the number of positions available for substituents on a ring (e.g., 2 positions available for (R^{V})ᵣ on U-11 in Exhibit 1), the actual higher end of the range is recognized to be the number of available positions.

When a compound is substituted with a substituent bearing a subscript that indicates the number of said substituents can exceed 1, said substituents (when they exceed 1) are independently selected from the group of defined substituents (e.g., (R^{V})ᵣ wherein r is 1, 2, 3, 4 or 5 in Exhibit 1). When a variable group is shown to be optionally attached to a position, for example (R^{V})ᵣ in Exhibit 1 wherein r may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 (e.g., substituent R², R³, or R⁴) is carbocyclic (e.g. phenyl) or heterocyclic (e.g. pyridinyl). The term “ring system” denotes two or more connected rings. The term “bicyclic ring system” denotes a ring system consisting of two rings sharing at least two common atoms. In a “fused bicyclic ring system” the common atoms are adjacent, and therefore the rings share two adjacent atoms and a bond connecting them (e.g., a pair of R⁷ substituents taken together to form a naphthalenyl ring system or a pair of R⁶ substituents taken together to form a fused ring). The term “spirocyclic ring system” denotes a ring system consisting of two rings connected at a single atom so the rings have a single atom in common. The term “ring member” refers to an atom (e.g., C, O, N or S) or other moiety (e.g., C(═O), C(═S) or S(═O)ₚ(═NR⁸)_{q}) forming the backbone of a ring or ring system.

The term “aromatic” indicates that each of the ring atoms of a fully unsaturated ring is essentially in the same plane and has a p-orbital perpendicular to the ring plane, and that (4n+2)π electrons, where n is a positive integer, are associated with the ring to comply with Hückel's rule.

The terms “carbocyclic ring”, “carbocycle” or “carbocyclic ring system” denote a ring or ring system wherein the atoms forming the ring backbone are selected only from carbon. Unless otherwise indicated, a carbocyclic ring can be a saturated, partially unsaturated or fully unsaturated ring. When a fully unsaturated carbocyclic ring satisfies Hückel's rule, then said ring is also called an “aromatic carbocyclic ring”. The term “saturated carbocyclic ring” refers to a ring having a backbone consisting of carbon atoms linked to one another by single bonds; unless otherwise specified, the remaining carbon valences are occupied by hydrogen atoms.

The terms “heterocyclic ring” or “heterocycle” denote rings in which at least one atom forming the ring backbone is not carbon (e.g., N, O or S). Unless otherwise indicated, a heterocyclic ring can be a saturated, partially unsaturated or fully unsaturated ring. The term “fully unsaturated heterocyclic ring” includes both aromatic and nonaromatic heterocycles. When a fully unsaturated heterocyclic ring satisfies Hückel's rule, then said ring is also called a “heteroaromatic ring” or “aromatic heterocyclic ring”. The terms “heteroaromatic ring system” or “heteroaromatic bicyclic ring system” denote a ring system in which at least one atom forming the ring backbone is not carbon (e.g., N, O or S) and at least one ring is aromatic. Unless otherwise indicated, heterocyclic rings and heteroaromatic ring systems can be attached through any available carbon or nitrogen by replacement of a hydrogen on said carbon or nitrogen.

In the context of the present invention when an instance of R², R³, or R⁴ comprises a phenyl ring or a 6-membered heterocyclic ring, the ortho, meta and para positions of each ring is relative to the connection of the ring to the remainder of Formula 1.

Naming of substituents in the present disclosure uses recognized terminology providing conciseness in precisely conveying to those skilled in the art the chemical structure. For sake of conciseness, locant descriptors may be omitted.

As noted above, each are R², R³, or R⁴ can independently be, inter alia, a phenyl ring optionally substituted with up to 5 substituents independently selected from the group of substituents as defined in the Summary of Invention. An example of a phenyl ring optionally substituted with up to five substituents is the ring illustrated as U-1 in Exhibit 1, wherein R^{V} is selected from a group of substituents as defined in the Summary of the Invention for R², R³, or R⁴ (i.e. R⁵, R⁶ and R⁷) and r is an integer from 0 to 5.

As noted above, each R², R³, or R⁴ can also be a 3- to 6-membered heterocyclic ring, each ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from any substituent as defined in the Summary of the Invention for R², R³, or R⁴ (i.e. R⁵, R^{5a}, R⁶, R^{6a}, R⁷ and R^{7a}). In this definition the substituents are optional, as 0 to 5 substituents may be present, limited only by the number of available points of attachment. The ring members selected from up to 2 O, up to 2 S and up to 3 N atoms are optional, provided at least one ring member is not carbon (e.g., N, O or S). The definition S(═O)ₚ(═NR⁸)_{q} allows the up to 2 sulfur atom ring members to be oxidized sulfur moieties (e.g., S(═O) or S(═O)₂) or unoxidized sulfur atoms (i.e. when p and q are both zero). The nitrogen atom ring members may be oxidized as N-oxides, because compounds relating to Formula 1 also include N-oxide derivatives. The up to 3 carbon atom ring members selected from C(═O) and C(═S) are in addition to the up to 4 heteroatoms selected from up to 2 O, up to 2 S and up to 3 N atoms. Examples of a 3-, 4-, 5- or 6-membered fully unsaturated heterocyclic ring include the rings U-2 through U-29 in Exhibit 1, and examples of a 3-, 4-, 5- or 6-membered saturated or partially unsaturated heterocyclic ring include the rings G-1 through G-45 in Exhibit 2. In Exhibits 1 and 2 the variable R^{V} is any substituent as defined in the Summary of the Invention for R², R³, or R⁴ (i.e. R⁵, R^{5a}, R⁶, R^{6a}, R⁷ and R^{7a}) and r is an integer from 0 to 5, limited by the number of available positions on each depicted ring. Although R^{V} groups are shown in the rings U-2 through U-29 and G-1 through G-45, it is noted that they do not need to be present since they are optional substituents. The nitrogen atoms that require substitution to fill their valence are substituted with H or R^{V}. Note that when the attachment point between (R^{V})ᵣ and the depicted ring is illustrated as floating, (R^{V})ᵣ can be attached to any available carbon or nitrogen atom of the depicted ring. Note that when the attachment point on the depicted ring is illustrated as floating, the depicted ring can be attached to the remainder of Formula 1 through any available carbon or nitrogen of the depicted ring by replacement of a hydrogen atom.

In Exhibit 2, note that when R², R³, or R⁴ comprises a ring selected from G-33, G-34, G-35 and G-40 through G-45, G² is O, S or N. Note that when G² is N, the nitrogen atom can complete its valence by substitution with either H or the substituents corresponding to R^{V} as defined in the Summary of the Invention for R², R³, or R⁴ (i.e. R^{5a}, R^{6a}, and R^{7a})

### Exhibit 1

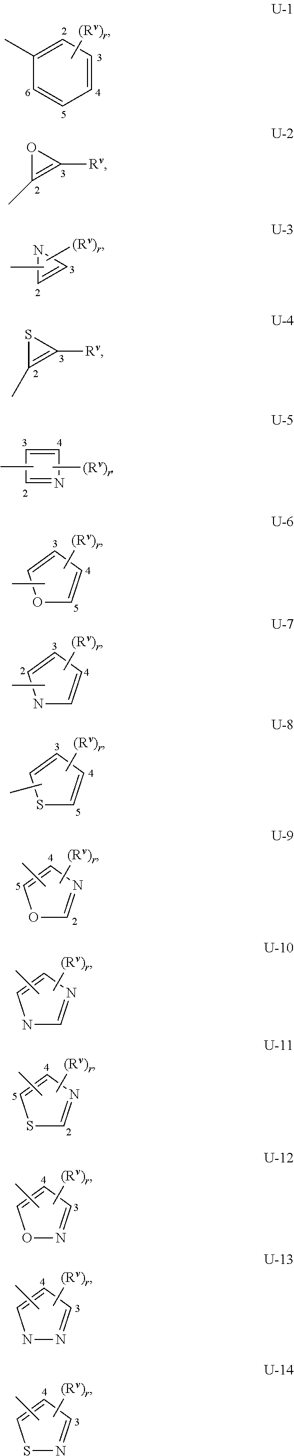

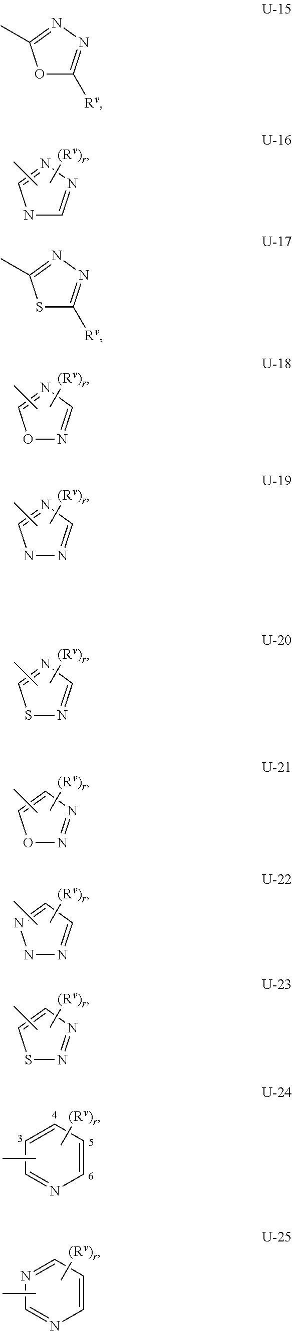

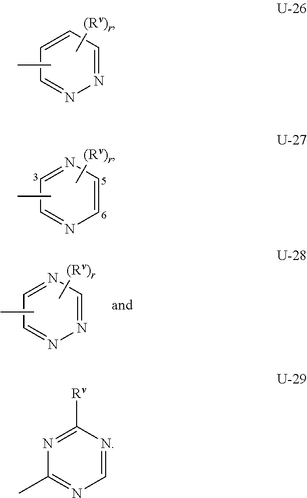

### Exhibit 2

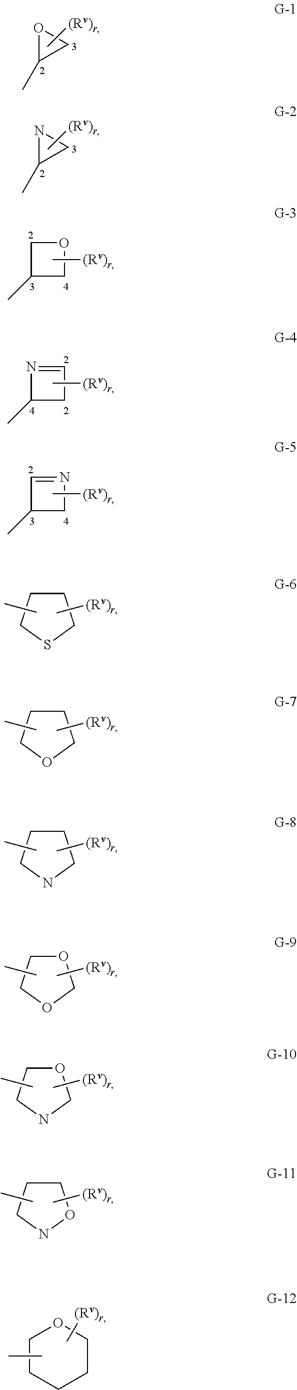

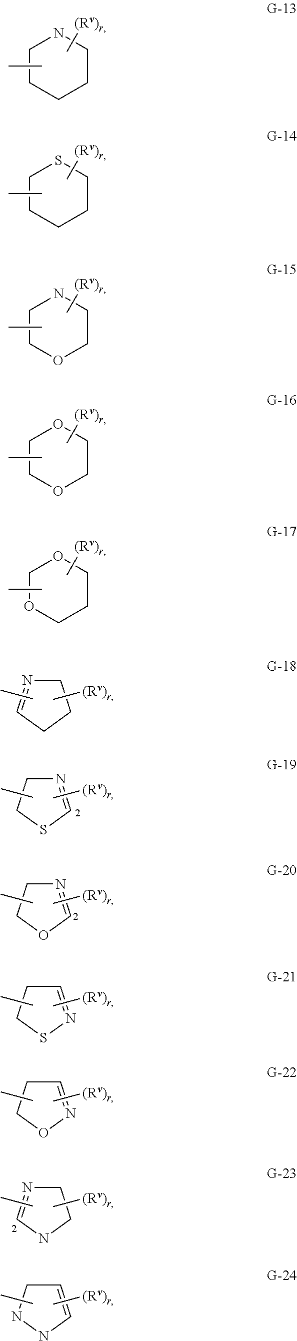

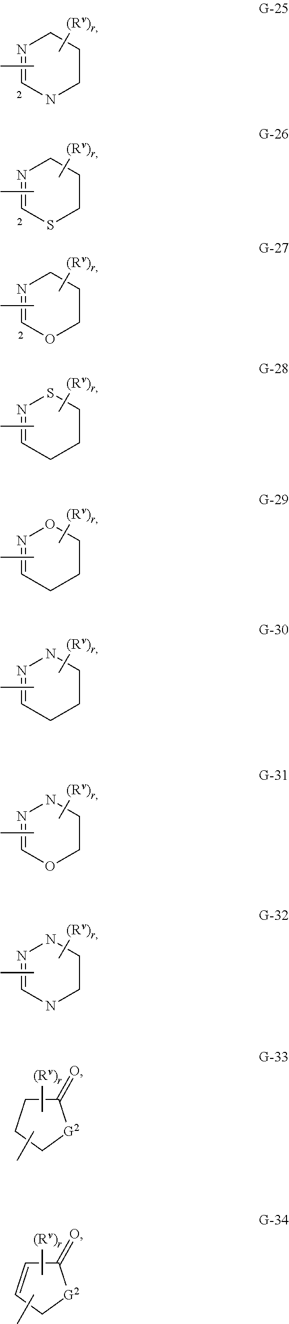

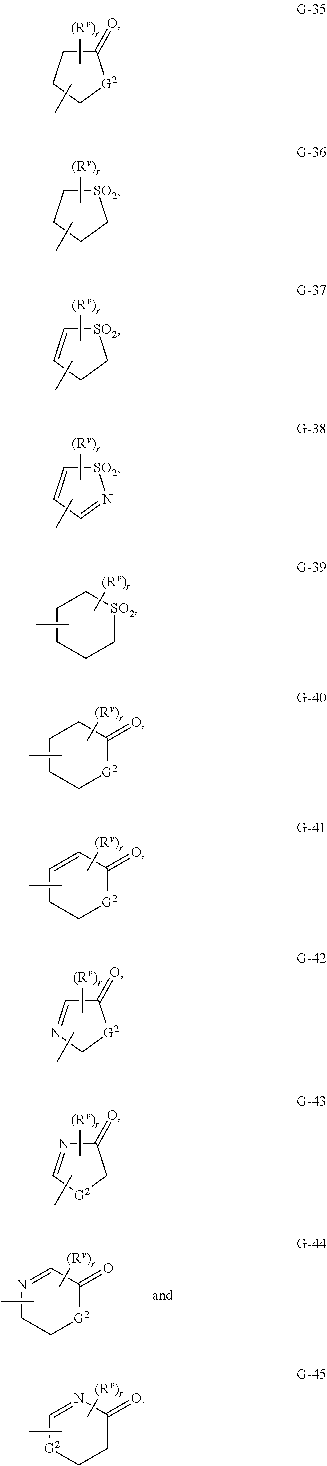

As noted in the Summary of the Invention, when a pair of R⁵, R^{5a}, R⁶, R^{6a} and R⁷ substituents are attached to adjacent ring atoms on Formula 1, besides the possibility of being separate substituents, they may also be connected to form a ring fused to the respective rings to which they are attached. The fused ring can be a 5-, 6- or 7-membered ring including as ring members the two atoms shared with the ring to which the substituents are attached. The other 3 to 5 ring members of the fused ring are provided by the pair of R⁵ substituents, the pair of R⁶ substituents, the pair of R⁵ and/or R^{5a} substituents or the pair of R⁶ and/or R^{6a} substituents taken together. These other ring members can include up to 5 carbon atoms (as allowed by the ring size) and optionally up to 4 heteroatoms selected from up to 2 O, up to 2 S and up to 3 N atoms. The fused ring is optionally substituted with up to 3 substituents as noted in the Summary of the Invention. Exhibit 3 provides, as illustrative examples, rings formed by a pair of adjacent R⁵, R⁶ or R⁷ substituents taken together. As these rings are fused with a ring of Formula 1, a portion of the Formula 1 ring is shown and the dashed lines represent the ring bonds of the Formula 1 ring. In certain cases, as illustrated by T-3, T-5, T-8, T-11, T-14 and T-16, the pattern of single and double bonds between ring members in the fused ring may affect the possible patterns of single and double bonds (according to valence bond theory) in the ring it is fused to in Formula 1, but each of the ring member atoms retains sp² hybridized orbitals (i.e. is able to participate in π-bonding). The rings depicted can be fused to any two adjacent atoms of a ring of Formula 1, and furthermore can be fused in either of the two possible orientations. The optional substituents (R^{V})ᵣ, are independently selected from halogen, cyano, nitro C₁-C₂ alkyl and C₁-C₂ alkoxy on carbon ring members and cyano C₁-C₂ alkyl and C₁-C₂ alkoxy on nitrogen ring members. For these T-rings, r is an integer from 0 to 3, limited by the number of available positions on each T-ring. When the attachment point between (R^{V})ᵣ and the T-ring is illustrated as floating, R^{V} may be bonded to any available T-ring carbon or nitrogen atom (as applicable). One skilled in the art recognizes that while r is nominally an integer from 0 to 3, some of the rings shown in Exhibit 3 have less than 3 available positions, and for these groups r is limited to the number of available positions. When “r” is 0 this means the ring is unsubstituted and hydrogen atoms are present at all available positions. If r is 0 and (R^{V})ᵣ is shown attached to a particular atom, then hydrogen is attached to that atom. The nitrogen atoms that require substitution to fill their valence are substituted with H or R^{V}. Furthermore, one skilled in the art recognizes that some of the rings shown in Exhibit 3 can form tautomers, and the particular tautomer depicted is representative of all the possible tautomers.

### Exhibit 3

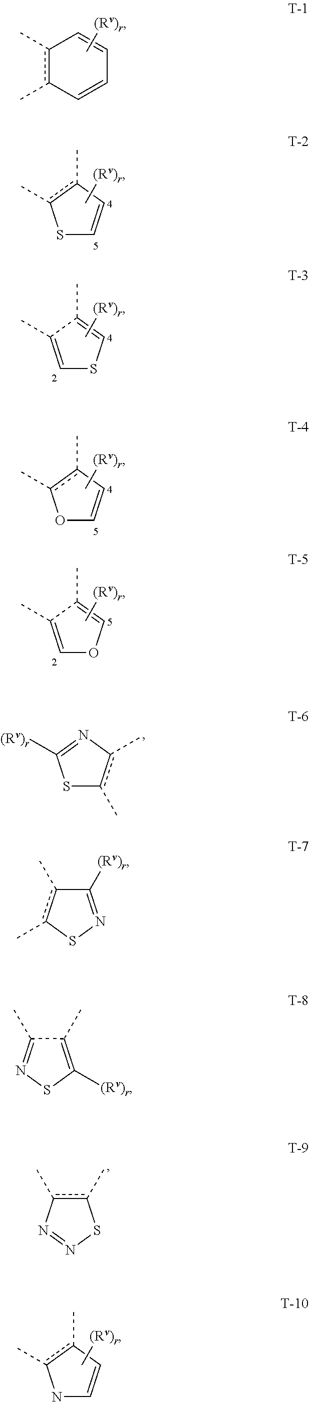

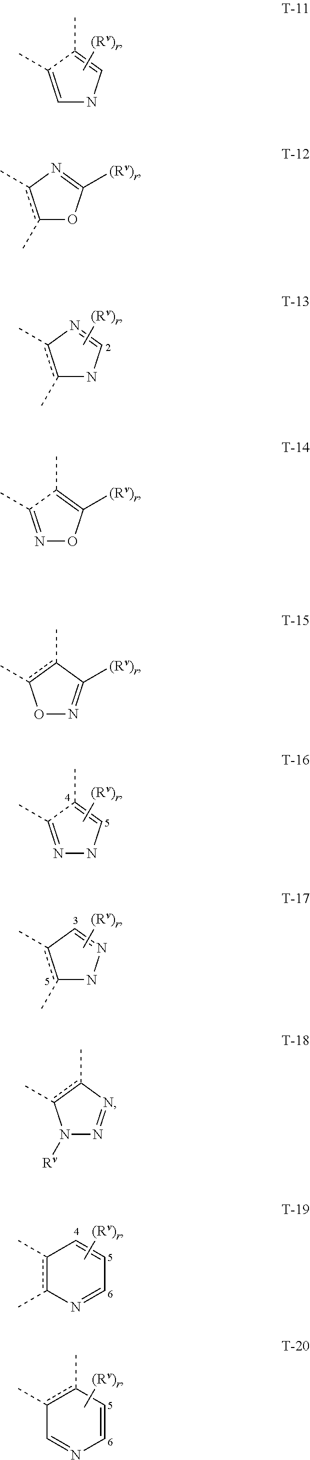

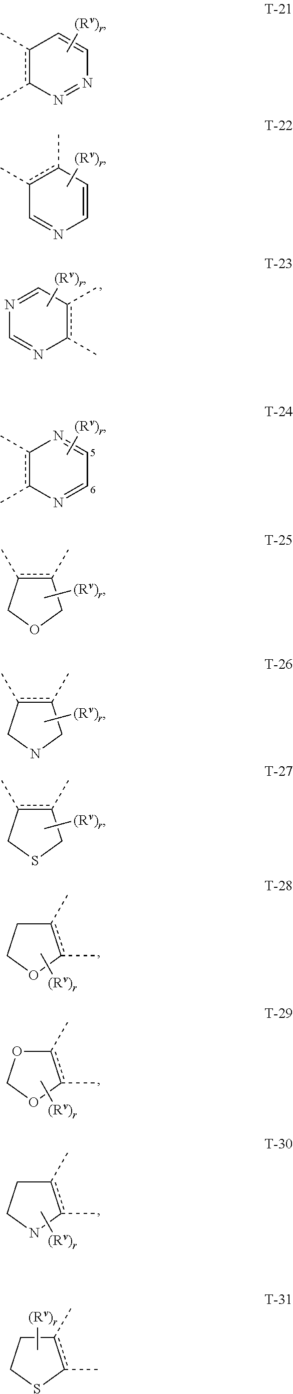

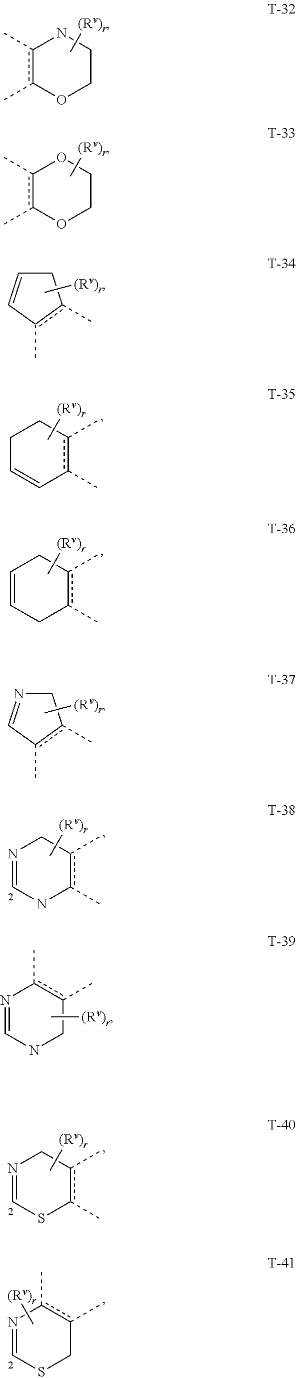

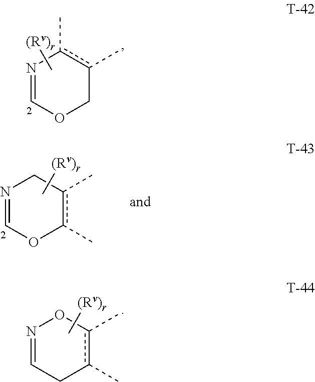

As noted in the Summary of the Invention, a pair of R⁵ or a pair of R⁶ substituents, besides the possibility of being separate substituents, may also be taken together with the carbon atom to which they are attached to form a 5-, 6- or 7-membered spirocyclic ring. The spirocyclic ring includes as a ring member the carbon atom shared with the ring to which the substituents are attached. The other 4 to 6 ring members of the spirocyclic ring are provided by the pair of R⁵ substituents or the pair of R⁶ substituents taken together. Exhibit 4 provides, as illustrative examples, rings formed by a pair of R⁵ or R⁶ substituents being taken together. As these rings are fused with a ring of Formula 1, a portion of the Formula 1 ring is shown and the dashed lines represent the ring bonds of the Formula 1 ring. The optional substituents (R^{V})ᵣ are independently selected from halogen, cyano, nitro C₁-C₂ alkyl and C₁-C₂ alkoxy on carbon ring members and cyano C₁-C₂ alkyl and C₁-C₂ alkoxy on nitrogen ring members. For these J-rings, r is an integer from 0 to 3, limited by the number of available positions on each J-ring. When the attachment point between (R^{V})ᵣ and the J-ring is illustrated as floating, R^{V} may be bonded to any available J-ring carbon or nitrogen atom. When “r” is 0 this means that the ring is unsubstituted and hydrogen atoms are present at all available positions.

### Exhibit 4

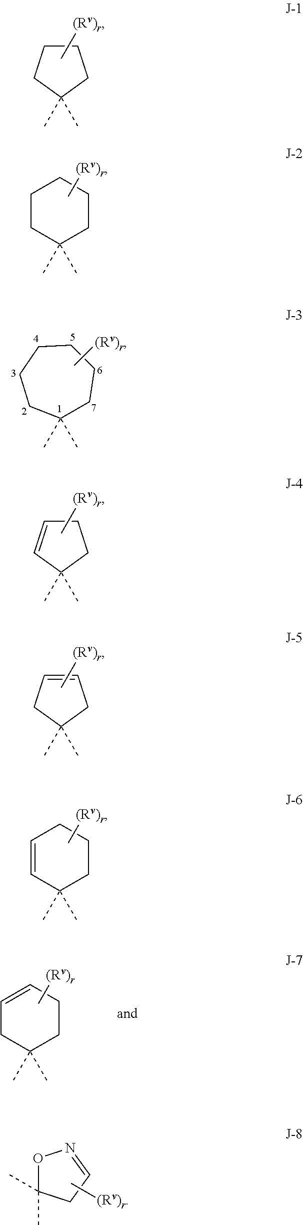

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

Compounds of this invention can form regioisomers. Regioisomers are compounds with the same molecular formula, but differ in their connectivity of the atoms or functional groups and result as an outcome of a chemical reaction. For example, in the present invention when the placement of an ring R² and R⁴ on Formula 1 are interchanged the resulting compounds are regioisomers. One skilled in the art will appreciate that one regioisomer may be more active and/or may exhibit beneficial effects when enriched relative to the other regioisomers or when separated from other regioisomers. Additionally, the skilled artisan knows how to separate, enrich, and/or to selectively prepare said regioisomers. The present invention includes compositions comprising regioisomers of Formula 1 in any ratio relative to one another. Typically said compositions comprise (a) at least one compound of Formula 1; and (b) at least one additional compound of Formula 1 which is a regioisomer of component (a); preferably the composition comprises on a weight basis at least about 50% of one regioisomer (i.e. one compound of Formula 1) relative to the sum of other regisomers present in the composition; and more preferably at least about 70% of one regioisomer; and most preferably at least about 95% of one regioisomer (for this calculation, the sum of the weight of the enriched regioisomer compound (including all stereoisomers) is divided by the sum of the weights of all other regioisomers present in the composition, and then the resulting division quotient is multiplied by 100%.

One skilled in the art will appreciate that not all nitrogen containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, Vol. 7, pp 748-750, S. V. Ley, Ed., Pergamon Press; M. Tisler and B. Stanovnik in Comprehensive Heterocyclic Chemistry, Vol. 3, pp 18-20, A. J. Boulton and A. McKillop, Eds., Pergamon Press; M. R. Grimmett and B. R. T. Keene in Advances in Heterocyclic Chemistry, Vol. 43, pp 149-161, A. R. Katritzky, Ed., Academic Press; M. Tisler and B. Stanovnik in Advances in Heterocyclic Chemistry, Vol. 9, pp 285-291, A. R. Katritzky and A. J. Boulton, Eds., Academic Press; and G. W. H. Cheeseman and E. S. G. Werstiuk in Advances in Heterocyclic Chemistry, Vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.

### Embodiment 1

A compound of Formula 1 wherein Q is S.

### Embodiment 2

A compound of Formula 1 wherein Q is O.

### Embodiment 3

A compound of Formula 1 or Embodiments 1 or 2 wherein Z is N.

### Embodiment 4

A compound of Formula 1 or Embodiments 1 or 2 wherein Z is CR¹.

### Embodiment 5

A compound of Embodiment 4 wherein R¹ is H.

### Embodiment 6

A compound of Embodiment 4 wherein R¹ is halogen, cyano, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ cyanoalkyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylamino or C₂-C₄ dialkylamino.

### Embodiment 7

A compound of Embodiment 6 wherein R¹ is C₁-C₄ alkyl, C₂-C₄ alkenyl, C₁-C₄ haloalkyl or C₁-C₃ alkoxy.

### Embodiment 8

A compound of Embodiment 7 wherein R¹ is C₁-C₄ alkyl.

### Embodiment 9

A compound of Embodiment 8 wherein R¹ is methyl, ethyl or n-propyl.

### Embodiment 10

A compound of Embodiment 9 wherein R¹ is methyl.

### Embodiment 11

A compound of Formula 1 or any one of Embodiments 1 through 10 wherein R² is a phenyl ring optionally substituted with up to 3 substituents independently selected from R⁵; or a 5- or 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the heterocyclic ring optionally substituted with up to 3 substituents independently selected from R⁵ on carbon atom ring members and R^{5a} on nitrogen atom ring members.

### Embodiment 12

A compound of Embodiment 11 wherein R² is a phenyl or pyridinyl ring optionally substituted with up to 3 substituents independently selected from R⁵.

### Embodiment 13

A compound of Embodiment 12 wherein R² is a phenyl ring optionally substituted with up to 3 substituents independently selected from R⁵.

### Embodiment 13a

A compound of Embodiment 13 wherein R² is a phenyl ring optionally substituted with up to 2 substituents independently selected from R⁵.

### Embodiment 13b

A compound of Embodiment 13 wherein R² is a phenyl ring substituted with 2 or 3 substituents independently selected from R⁵.

### Embodiment 14

A compound of Formula 1 or any one of Embodiments 1 through 13b wherein when R² is an optionally substituted phenyl or pyridinyl ring, then R² is substituted with 2 or 3 substituents.

### Embodiment 15

A compound of Formula 1 or any one of Embodiments 1 through 14 wherein when R² is an optionally substituted phenyl ring, then R² is substituted with 2 or 3 substituents.

### Embodiment 16

A compound of Formula 1 or any one of Embodiments 1 through 15 wherein when R² is an optionally substituted phenyl ring, then R² is substituted with 2 substituents.

### Embodiment 17

A compound of Formula 1 or any one of Embodiments 1 through 16 wherein when R² is an optionally substituted phenyl ring, then R² is substituted with at least two substituents at the meta positions.

### Embodiment 18

A compound of Formula 1 or any one of Embodiments 1 through 17 wherein when R² is an optionally substituted phenyl ring, then R² is substituted with two substituents at the meta positions.

### Embodiment 19

A compound of Formula 1 or any one of Embodiments 1 through 16 wherein when R² is an optionally substituted phenyl ring, then R² is substituted with two substituents at the ortho positions.

### Embodiment 20

A compound of Formula 1 or any one of Embodiments 1 through 15 wherein when R² is an optionally substituted phenyl ring, then R² is substituted with two substituents at the meta positions and one substituent at an ortho position.

### Embodiment 21

A compound of Formula 1 or any one of Embodiments 1 through 20 wherein R³ is a phenyl ring optionally substituted with up to 3 substituents independently selected from R⁶; or a 5- or 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the heterocyclic ring optionally substituted with up to 3 substituents independently selected from R⁶ on carbon atom ring members and R^{6a} on nitrogen atom ring members; or halogen, cyano, nitro, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy or C₃-C₆ cycloalkoxy.

### Embodiment 22

A compound of Embodiment 21 wherein R³ is a phenyl ring optionally substituted with up to 3 substituents independently selected from R⁶; or a 5- or 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the heterocyclic ring optionally substituted with up to 3 substituents independently selected from R⁶ on carbon atom ring members and R^{6a} on nitrogen atom ring members; or halogen, cyano, nitro, C₁-C₆ alkyl, C₂-C₆ alkenyl, C₂-C₆ alkynyl, C₁-C₆ haloalkyl, C₂-C₆ haloalkenyl, C₂-C₆ haloalkynyl or C₃-C₆ cycloalkyl.

### Embodiment 23

A compound of Embodiment 22 wherein R³ is a phenyl or pyridinyl ring optionally substituted with up to 3 substituents independently selected from R⁶; or halogen or C₁-C₆ alkyl.

### Embodiment 24

A compound of Embodiment 23 wherein R³ is a phenyl ring optionally substituted with up to 3 substituents independently selected from R⁶; or C₁-C₆ alkyl.

### Embodiment 25

A compound of Embodiment 24 wherein R³ is a phenyl ring optionally substituted with up to 3 substituents independently selected from R⁶; or methyl or i-propyl.

### Embodiment 25a

A compound of Embodiment 25 wherein R³ is a phenyl ring optionally substituted with up to 3 substituents independently selected from R⁶; or methyl.

### Embodiment 25a

A compound of Embodiment 25 wherein R³ is a phenyl ring substituted with 1, 2 or 3 substituents independently selected from R⁶; or methyl.

### Embodiment 25b

A compound of Embodiment 25 wherein R³ is methyl.

### Embodiment 25c

A compound of Embodiment 25 wherein R³ is a phenyl ring optionally substituted with up to 3 substituents.

### Embodiment 26

A compound of Embodiment 25 wherein R³ is a phenyl ring optionally substituted with up to 2 substituents independently selected from R⁶; or methyl or i-propyl.

### Embodiment 26a

A compound of Embodiment 26 wherein R³ is a phenyl ring optionally substituted with up to 2 substituents independently selected from R⁶; or methyl.

### Embodiment 26b

A compound of Embodiment 26a wherein R³ is a phenyl ring optionally substituted with up to 2 substituents.

### Embodiment 27

A compound of Embodiment 25 wherein R³ is a phenyl ring optionally substituted with up to 1 substituent selected from R⁶; or methyl.

### Embodiment 27a

A compound of Embodiment 27 wherein R³ is a phenyl ring optionally substituted with up to 1 substituent selected from R⁶.

### Embodiment 27b

A compound of Embodiment 27a wherein R³ is a phenyl ring.

### Embodiment 28

A compound of Formula 1 or any one of Embodiments 1 through 27b wherein when R³ is an optionally substituted phenyl or pyridinyl ring, then R³ is substituted with 2 or 3 substituents.

### Embodiment 29

A compound of Formula 1 or any one of Embodiments 1 through 28 wherein when R³ is an optionally substituted phenyl ring, then R³ is substituted with 2 substituents.

### Embodiment 30

A compound of Formula 1 or any one of Embodiments 1 through 29 wherein when R³ is an optionally substituted phenyl ring, then R³ is substituted with 3 substituents.

### Embodiment 31

A compound of Formula 1 or any one of Embodiments 1 through 30 wherein when R³ is an optionally substituted phenyl ring, then R³ is substituted with at least one substituent at an ortho position.

### Embodiment 32

A compound of Formula 1 or any one of Embodiments 1 through 31 wherein when R³ is an optionally substituted phenyl ring, then R³ is substituted with three substituents at the ortho and para positions.

### Embodiment 33

A compound of Formula 1 or any one of Embodiments 1 through 32 wherein R⁴ is a phenyl ring optionally substituted with up to 3 substituents independently selected from R⁷; or a 5- or 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the heterocyclic ring optionally substituted with up to 3 substituents independently selected from R⁷ on carbon atom ring members and R^{7a} on nitrogen atom ring members; or
halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy or C₃-C₆ cycloalkoxy.

### Embodiment 34

A compound of Embodiment 33 wherein R⁴ is a phenyl ring optionally substituted with up to 3 substituents independently selected from R⁷; or a 5- or 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the heterocyclic ring optionally substituted with up to 3 substituents independently selected from R⁷ on carbon atom ring members and R^{7a} on nitrogen atom ring members; or halogen, cyano, nitro, C₁-C₆ alkyl, C₂-C₆ alkenyl, C₂-C₆ alkynyl, C₁-C₆ haloalkyl, C₂-C₆ haloalkenyl, C₂-C₆ haloalkynyl or C₃-C₆ cycloalkyl.

### Embodiment 35

A compound of Embodiment 34 wherein R⁴ is a phenyl or pyridinyl ring optionally substituted with up to 3 substituents independently selected from R⁷; or halogen or C₁-C₆ alkyl.

### Embodiment 36

A compound of Embodiment 35 wherein R⁴ is a phenyl ring optionally substituted with up to 3 substituents independently selected from R⁷; or C₁-C₆ alkyl.

### Embodiment 37

A compound of Embodiment 36 wherein R⁴ is a phenyl ring optionally substituted with up to 3 substituents independently selected from R⁷; or methyl.

### Embodiment 38

A compound of Embodiment 37 wherein R⁴ is a phenyl ring substituted with 1, 2 or 3 substituents independently selected from R⁷; or methyl.

### Embodiment 38a

A compound of Embodiment 38 wherein R⁴ is methyl.

### Embodiment 38b

A compound of Embodiment 38 wherein R⁴ is a phenyl ring substituted with 3 substituents independently selected from R⁷.

### Embodiment 38c

A compound of Embodiment 38 wherein R⁴ is a phenyl ring substituted with 2 substituents independently selected from R⁷.

### Embodiment 39

A compound of Embodiment 37 wherein R⁴ is a phenyl ring optionally substituted with up to 2 substituents independently selected from R⁷; or methyl.

### Embodiment 40

A compound of Embodiment 37 wherein R⁴ is a phenyl ring.

### Embodiment 41

A compound of Formula 1 or any one of Embodiments 1 through 40 wherein when R⁴ is an optionally substituted phenyl ring, then R⁴ is substituted with 3 substituents.

### Embodiment 42

A compound of Formula 1 or any one of Embodiments 1 through 40b wherein when R⁴ is an optionally substituted phenyl ring, then R⁴ is substituted with 2 substituents.

### Embodiment 43

A compound of Formula 1 or any one of Embodiments 1 through 42 wherein when R⁴ is an optionally substituted phenyl ring, then R⁴ is substituted with at least one substituent at an ortho position.

### Embodiment 44

A compound of Formula 1 or any one of Embodiments 1 through 43 wherein when R⁴ is an optionally substituted phenyl ring, then R⁴ is substituted with one substituent at an ortho position and one at the para position.

### Embodiment 45

A compound of Formula 1 or any one of Embodiments 1 through 44 wherein each R⁵, R⁶ and R⁷ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.

### Embodiment 46

A compound of Embodiment 45 wherein each R⁵, R⁶ and R⁷ is independently halogen, C₁-C₆ alkyl, C₂-C₆ alkenyl, C₁-C₆ haloalkyl or C₁-C₆ alkoxy.

### Embodiment 47

A compound of Embodiment 46 wherein each R⁵, R⁶ and R⁷ is independently halogen, C₁-C₃ alkyl or C₁-C₃ alkoxy.

### Embodiment 48

A compound of Embodiment 47 wherein each R⁵, R⁶ and R⁷ is independently halogen, methyl or methoxy.

### Embodiment 49

A compound of Embodiment 48 wherein each each R⁵, R⁶ and R⁷ is independently chloro, fluoro or methoxy.

### Embodiment 50

A compound of Embodiment 49 wherein each R⁵ is independently chloro or methoxy.

### Embodiment 51

A compound of Embodiment 49 wherein each R⁶ is independently chloro or fluoro.

### Embodiment 52

A compound of Embodiment 49 wherein each R⁷ is independently fluoro or methoxy.

### Embodiment 53

A compound of Formula 1 or any one of Embodiments 1 through 52 wherein each R^{5a}, R^{6a} and R^{7a} is independently cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.

### Embodiment 54

A compound of Embodiment 53 wherein each R^{5a}, R^{6a} and R^{7a} is independently C₁-C₆ alkyl, C₂-C₆ alkenyl, C₁-C₆ haloalkyl or C₁-C₆ alkoxy.

### Embodiment 55

A compound of Embodiment 54 wherein each R^{5a}, R^{6a} and R^{7a} is independently C₁-C₂ alkyl.

### Embodiment 56

A compound of Formula 1 or any one of Embodiments 1 through 55 wherein each R⁸ is independently H or methyl.

### Embodiment 57

A compound of Embodiment 56 wherein each R⁸ is independently H.

Embodiments of this invention, including Embodiments 1-57 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-57 above as well as any other embodiments described herein, and any combination thereof, pertain to the compositions and methods of the present invention.

Combinations of Embodiments 1-57 are illustrated by:

### Embodiment A1

A compound of Formula 1 wherein:
Q is O;
R¹ is C₁-C₄ alkyl, C₂-C₄ alkenyl, C₁-C₄ haloalkyl or C₁-C₃ alkoxy;
R² is a phenyl or pyridinyl ring optionally substituted with up to 3 substituents independently selected from R⁵;
R³ is a phenyl or pyridinyl ring optionally substituted with up to 3 substituents independently selected from R⁶; or halogen or C₁-C₆ alkyl;
R⁴ is a phenyl or pyridinyl ring optionally substituted with up to 3 substituents independently selected from R⁷; or halogen or C₁-C₆ alky; and
each R⁵, R⁶ and R⁷ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.

### Embodiment A2

A compound of Embodiment A1 wherein
R¹ is C₁-C₄ alkyl;
R² is a phenyl ring optionally substituted with up to 3 substituents independently selected from R⁵;
R³ is a phenyl ring optionally substituted with up to 3 substituents independently selected from R⁶; or methyl or i-propyl;
R⁴ is a phenyl ring optionally substituted with up to 3 substituents independently selected from R⁷; or methyl; and
each R⁵, R⁶ and R⁷ is independently halogen, C₁-C₆ alkyl, C₂-C₆ alkenyl, C₁-C₆ haloalkyl or C₁-C₆ alkoxy.

### Embodiment A3

A compound of Embodiment A2 wherein
R² is a phenyl ring substituted with 2 or 3 substituents independently selected from R⁵;
R³ is a phenyl ring substituted with 1, 2 or 3 substituents independently selected from R⁶; or methyl;
R⁴ is a phenyl ring substituted with 1, 2 or 3 substituents independently selected from R⁷; or methyl; and
each R⁵, R⁶ and R⁷ is independently halogen, methyl or methoxy.

### Embodiment A4

A compound of Embodiment A3 wherein:
Z is CR¹;
R¹ is methyl;
R⁴ is a phenyl ring substituted with 2 or 3 substituents independently selected from R⁷; and
each R⁵, R⁶ and R⁷ is independently chloro, fluoro or methoxy.

### Embodiment A5

A compound of Embodiment A3 wherein:
Z is N;
R³ is a phenyl ring substituted with 1, 2 or 3 substituents independently selected from R⁶;
R⁴ is methyl; and
each R⁵, R⁶ and R⁷ is independently chloro, fluoro or methoxy.

Specific embodiments include compounds of Formula 1 selected from the group consisting of:
4-(2-chloro-3,5-dimethoxyphenyl)-5-(2-chloro-4-fluorophenyl)-3,6-dimethyl-2H-pyran-2-one;
5-(2,4-difluorophenyl)-4-(3,5-dimethoxyphenyl)-3,6-dimethyl-2H-pyran-2-one;
5-(2,4-difluorophenyl)-4-(3,5-dimethoxyphenyl)-3-(2-fluorophenyl)-6-methyl-2H-pyran-2-one;
4-(3,5-dimethoxyphenyl)-3-(2-fluorophenyl)-5-(2,4,6-trifluorophenyl)-6H-1,2-oxazin-6-one; and
4-(2-chloro-3,5-dimethoxyphenyl)-3-methyl-5-(2,4,6-trifluorophenyl)-6H-1,2-oxazin-6-one.

Embodiments of the present invention as described in the Summary of the Invention also include those described below. In the following Embodiments, Formula 1 includes stereoisomers, N-oxides and salts thereof, and reference to “a compound of Formula 1” includes the definitions of substituents specified in the Summary of the Invention unless further defined in the Embodiments.

### Embodiment 1B

The composition described in the Summary of the Invention comprising (a) at least one compound of Formula 1 wherein Q is O, Z is CR¹, R¹ is methyl, R² is 3-methoxyphenyl, 3,5-dimethoxyphenyl or 2-chloro-3,5-dimethoxyphenyl, R³ is methyl and R⁴ is 2,6-difluorophenyl, 2,4-difluorophenyl, 2-chloro-4-fluorophenyl, 2,4,6-trifluorophenyl or 2,6-difluoro-4-methoxyphenyl; and (b) at least one additional compound of Formula 1 which is a regisomer of component (a) wherein Q is O, Z is CR¹, R¹ is methyl, R² is 2,6-difluorophenyl, 2,4-difluorophenyl, 2-chloro-4-fluorophenyl, 2,4,6-trifluorophenyl or 2,6-difluoro-4-methoxyphenyl, R³ is methyl and R⁴ is 3-methoxyphenyl, 3,5-dimethoxyphenyl or 2-chloro-3,5-dimethoxyphenyl.

### Embodiment 2B

A composition of Embodiment 1B wherein (a) Q is O, Z is CR¹, R¹ is methyl, R² is 2-chloro-3,5-dimethoxyphenyl, R³ is methyl and R⁴ is 2-chloro-4-fluorophenyl; and (b) Q is O, Z is CR¹, R¹ is methyl, R² is 2-chloro-4-fluorophenyl, R³ is methyl and R⁴ is 2-chloro-3,5-dimethoxyphenyl.

### Embodiment 3B

A composition of Embodiment 1B wherein (a) Q is O, Z is CR¹, R¹ is methyl, R² is 2-chloro-3,5-dimethoxyphenyl, R³ is methyl and R⁴ is 2,4,6-trifluorophenyl; and (b) Q is O, Z is CR¹, R¹ is methyl, R² is 2,4,6-trifluorophenyl, R³ is methyl and R⁴ is 2-chloro-3,5-dimethoxyphenyl.

### Embodiment 4B

The composistion of anyone of Embodiments 1B through 3B comprising on a weight basis at least about 50% of component (a) relative to the sum of the weight of component (b).

### Embodiment 5B

The composistion of Embodiment 4B comprising on a weight basis at least about 70% of component (a) relative to the sum of the weight of component (b).

### Embodiment 5B

The composistion of Embodiment 4B comprising on a weight basis at least about 95% of component (a) relative to the sum of the weight of component (b).

This invention provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention further provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention also provides a fungicidal composition comprising a compound of Formula 1 (including all stereoisomers, N-oxides, and salts thereof) (i.e. in a fungicidally effective amount), and at least one additional compound of Formula 1 (i.e. in a fungicidally effective amount) which is a regisomer of the first compound. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as an embodiment of such methods are methods comprising applying a fungicidally effective amount of a compound corresponding to any of the compound embodiments described above. Of particular note are embodiments where the compounds are applied as compositions of this invention.

One or more of the following methods and variations as described in Schemes 1-10 can be used to prepare the compounds of Formula 1. The definitions of Q, Z, R¹, R², R³ and R⁴ in the compounds of Formulae 1-15 below are as defined above in the Summary of the Invention unless otherwise noted. Compounds of Formulae 1a-1d are various subsets of the compounds of Formula 1, and all substituents for Formulae 1a-1d are as defined above for Formula 1 unless otherwise noted.

Oxazinones of Formula 1a (Formula 1 wherein Z is N and W is O) can be synthesized by condensation of furanones Formula 2 with hydroxylamine or a hydroxylamine salt (e.g., hydroxylamine hydrochloride) as shown in Scheme 1. The reaction is typically run in a lower alkanol solvent, such as methanol, ethanol, n-butanol, methanol-water or ethanol-water mixtures at a temperature ranging from about room temperature to the reflux temperature of the solvent. The presence of an acid acceptor such as an alkali hydroxide or alkali carbonate can also be beneficial to the outcome of the reaction particularly when the hydroxylamine is present in salt form. Upon completion of the reaction, the reaction is poured into water, and the isolated intermediate is treated with an acid such as glacial acetic acid to provide the compound of Formula 1a. In a preferred method, the isolated intermediate is heated with glacial acetic acid as the solvent to a temperature of about 110° C. Example 1, Step A and illustrates the preparation of a compound of Formula 1a using the method of Scheme 1.

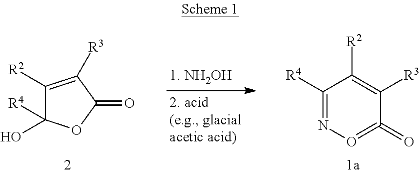

Compounds of Formula 2 are known in the literature, for relevant references see the following: Patent PCT Patent Application Publication WO 2010/036553, WO 2009/090039, WO 2008/009406, WO 2008/009405, WO 2007/080720, WO 2007/066601 and WO 2005/121104 and European Patent Application Publications EP1978023A1, EP1974608A1, EP1972623A1 and EP1916240A1.

Pyranones of Formula 1b (Formula 1 wherein Z is CR¹ and W is O) can be prepared by the reaction of alkynes of Formula 3 with acetoacetate derivatives of Formula 4 in the presence of a rhenium catalyst as shown in Scheme 2. In some cases, depending on the reaction conditions, these reactions result in mixtures regioisomers, for example, where R² and R⁴ on Formula 1 are interchanged. The reaction can be performed in a variety of solvents, such as examples toluene, xylenes, N,N-dimethylformamide, N,N-dimethylacetamide, dimethylsulfoxide or N-methylpyrrolidinone. The reaction can be carried out with conventional or microwave heating at temperatures ranging from about 60 to 200° C. Preferably, the rhenium catalyst is Re(CO)Br(thf)₂ and is present in 1 to 10% molar quantity relative to the substrate. For conditions and variations of this reaction see the following references: see Kuninobu et al., Chemical Communications 2008, 50, 6360-6362 and Journal of Organic Chemistry 2010, 75, 334-341.

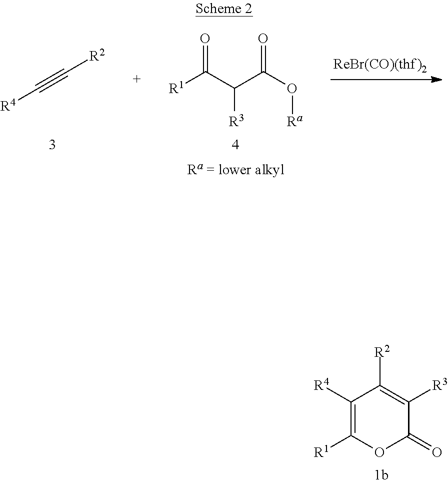

Alternatively, pyranones of Formula 1b can be prepared using well-known transition metal-catalyzed cross coupling reactions as shown in Scheme 3. As illustrated, pyrones of Formula 5 wherein X leaving group such as halogen (e.g., Cl, Br, I), sulfonate (e.g., OS(O)₂CH₃, OS(O)₂CF₃, OS(O)₂Ph-p-CH₃), and the like, can be reacted with organometallic reagents R¹M¹ (Formula 6) in the presence of palladium, nickel, iron or copper catalysts to provide pyranones of Formula 1b. In this method compounds of Formula 6 are organoboronic acids (e.g., M is B(OH)₂), organotrifluoroborates (e.g., M is BF₃K), organotin reagents (e.g., M is Sn(n-Bu)₃, Sn(Me)₃), Grignard reagents (e.g., M is MgX¹) or organozinc reagents (e.g., M is ZnX¹) wherein X¹ is I, Br or Cl. Suitable transition metal catalysts include but are not limited to palladium(II) acetate, palladium(II) chloride, tetrakis-(triphenylphosphine)palladium(0), bis(triphenylphosphine)palladium(II) dichloride, dichloro[1,1′-bis(diphenylphosphino)ferrocene]palladium(II), bis(triphenylphosphine)dichloro-nickel(II), copper(I) salts (e.g., copper(I) iodide, copper(I) bromide, copper(I) chloride, copper(I) cyanide and copper(I) triflate) and iron(III) acetylacetonate. Optimal conditions for each reaction will depend upon the catalyst used and the counterion associated with the compound of Formula 6 (i.e. M), as is understood by one skilled in the art. In some cases the addition of a ligand such as a substituted phosphine or a substituted bisphosphinoalkane promoting an increase in the reaction rate. Also, the presence of a base (such as an alkali carbonate, tertiary amine or alkali fluoride) is typically necessary for reactions involving compounds of Formula 6 where M is a boronic acid or organotrifluoroborate. For reviews of this type of reaction, see: E. Negishi, Handbook of Organopalladium Chemistry for Organic Synthesis, John Wiley and Sons, Inc., New York, 2002; N. Miyaura, Cross-Coupling Reactions: A Practical Guide, Springer, N.Y., 2002; H. C. Brown et al., Organic Synthesis via Boranes, Vol. 3, Aldrich Chemical Co., Milwaukee, Wis., 2002; Suzuki et al., Chemical Review 1995, 95(7), 2457-2483 and Molander et al., Accounts of Chemical Research 2007, 40, 275-286.

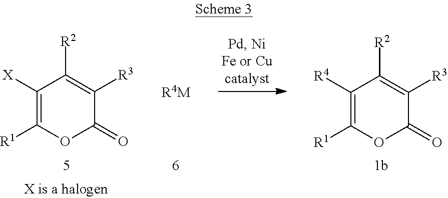

One skilled in the art will recognize that depending on the availability of starting materials and/or how other functionalities that may be present in compounds of Formulae 5 and 6 can effect the outcome of the reaction, it may be more advantageous to perform the method analogous to Scheme 3 by reacting a pyrone analogous to Formula 5 wherein the ring is substituted with R⁴, X and R³ and an organometallic reagent analogous to Formula 6 containing R² to provide compounds of Formula 1b as illustrated in Scheme 4 below.

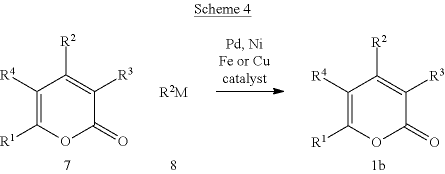

Compounds of Formula 5 can be prepared by halogenation of pyrones of Formula 9 as shown in Scheme 5. Typically halogenation can be achieved using a variety of halogenating reagents known in the art such as elemental halogen (e.g., Cl₂, Br₂, I₂), sulfuryl chloride, iodine monochloride or a N-halosuccinimide (e.g., NBS, NCS, NIS) in an appropriate solvent such as N,N-dimethylformamide, carbon tetrachloride, acetonitrile, dichloromethane or acetic acid. Typically the method of Scheme 5 is run using N-halosuccinimides in a polar solvent (e.g., N,N-dimethylformamide, acetonitrile, methanol, ethanol or i-propanol) at temperatures ranging from about 0 to 100° C.

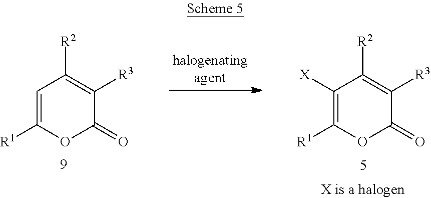

Compounds of Formula 9 can be prepared by the reaction of alkynes of Formula 10 with acetoacetate derivatives of Formula 4 in the presence of a rhenium or manganese catalyst as shown in Scheme 6. The reaction can be performed in a variety of solvents. Some examples include aromatic hydrocarbons such as toluene and xylenes, N,N-dimethyl-formamide, N,N-dimethylacetamide, dimethylsulfoxide or N-methylpyrrolidinone. The reaction can be carried out with conventional or microwave heating at temperatures ranging from about 60 to 200° C. Preferably, the rhenium catalyst is Re(CO)Br(thf)₂ and is present in 1 to 10% molar quantity compared to the substrate. Other catalysts such as MnBr(CO)₅ can also be used under similar conditions. A fluoride source such as tetrabutylammonium fluoride can be added to the reaction to improve the yield of a compound of Formula 9. For more details on rhenium catalyzed pyrone synthesis see Kuninobu et al., Chemical Communications 2008, 50, 6360-6362 and Journal of Organic Chemistry 2010, 75, 334-341.

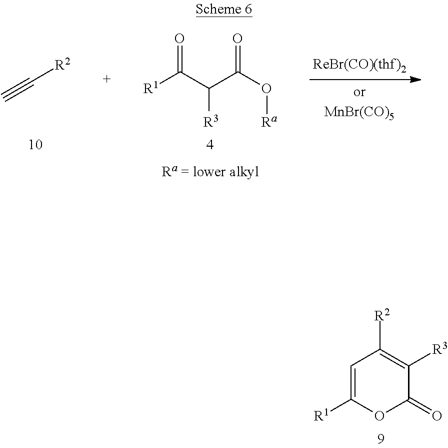

Compounds of Formula 7 can be prepared by reaction of pyrones of Formula 11 with a halogenation reagent as shown in Scheme 7. Typical examples of halogenating reagents which are useful for this reaction include, but are not limited to, phosphorous oxychloride, phosphorus oxybromide, phosphorous pentachloride, phosphorous triiodide, and phosphorous tribromide. The halogenation reaction may be carried out neat or with a solvent that does not react with the halogenation reagent including, but not limited to, diethyl ether, toluene, dichloromethane, dichlorethane, or benzene. The reaction can be carried out at temperatures between about −20° C. and 180° C. Additives such as N,N-dimethylformamide can facilitate this reaction. In some cases addition of a tertiary amine base may also be of benefit. For examples of halogenation of 4-hydroxypyrones in the literature, see Fairlamb et al., Bioorganic and Medicinal Chemistry 2004, 12, 4285-4299 (with PBr₃ and N,N-dimethylformamide), and Hormi et al., Journal of Organic Chemistry 1987, 52, 5275-5276 (with POCl₃).

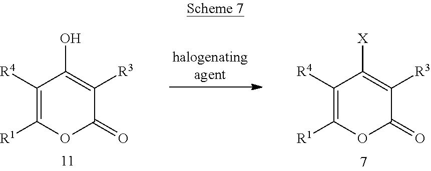

As shown in Scheme 8, compounds of Formula 11 may be prepared by the cyclization of malonyl dichlorides of Formula 12 with ketones of Formula 13. Typical reaction conditions involve heating compounds of Formula 12 and Formula 13 in a suitable solvent at a temperature ranging from about 20 to 220° C. Suitable solvents include, but are not limited to, aromatic hydrocarbons such as benzene, toluene, diethyl benzene, mesitylene and xylenes; aliphatic hydrocarbons such as decalin, and chlorinated aromatic hydrocarbons such as chlorobenzene and o-dichlorobenezene. The reaction may also be carried out in the absence of solvent. Depending on the conditions of the reaction, the actual activated malonate may be in the form of the malonyldichloride or the 2-chlorocarbonylketene, both leading to the same compounds. Some examples of pyrone syntheses via cyclizations of activated malonates can be found in U.S. Pat. Nos. 5,393,729, 5,977,029, 6,071,937, 6,140,358 and 6,417,370, and International Patent Publication WO 2001/074770. See Lieb et al., Tetrahedron 2001, 57, 4133-4137 for similar chemistry using the silyl enol ethers of the ketones. Ketones of Formula 13 are commercially available or can be prepared by methods outlined in Patent Application Publications WO 2009/158257 and WO 2005/121104. Malonic acid precursors of compounds of Formula 12 can be made by procedures outlined in U.S. Pat. Nos. 5,393,729, 5,977,029, 6,071,937, 6,140,358, and 6,417,370; and in Patent Application Publications WO 2009/099929 and WO 2001/074770.

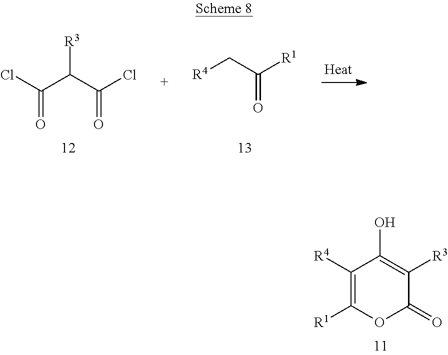

Alkynes of Formula 3 and Formula 10 are commercially available and can be prepared using the using well-known Sonogashira reaction conditions. The reaction typically involves the use of two catalysts, a zero-valent palladium complex (or one that can be reduced to Pd(0) in situ) and a halide salt of copper(I). Useful catalysts for this type of transformation include tetrakis(triphenylphosphine)palladium(0), bis(triphenylphosphine)-palladium(II) chloride, dichlorobis(tri-o-tolylphosphine)palladium, copper(I) iodide, copper(I) bromide and copper(I) chloride. Suitable solvents include amines (e.g., triethylamine or diethylamine), or solvents such as tetrahydrofuran, acetonitrile, ethyl acetate and N,N-dimethylformamide used in combination with a large excess of a base including, for example, triethylamine, diethylamine, potassium carbonate or cesium carbonate. The general method is taught by Campbell, Organocopper Reagents 1994, 217-235; Sonogashira et al., Tetrahedron Letters 1975, 50, 4467-4470; Chinchilla et al., Chemical Review 2007, 107, 874-922 and Kosower et al., Journal of Organic Chemistry 1996, 61(17), 5871-5884.

Alternatively, alkynes of Formula 3 and Formula 10 can be synthesis using the Colvin rearrangement is shown in Scheme 9. Reaction of ketones of Formula 14 with lithiated trimethyldiazomethane gives compounds of Formula 10 and likewise reaction of aldehydes of Formula 15 gives terminal alkynes of Formula 3. A solution of trimethylsilyldiazomethane is lithiated with a strong base such as n-butyllithium or lithium diisopropylamide and treated with the ketone or aldehyde at about −20 to −80° C. The reaction is then heated to about 20 to 80° C. The reaction is typically carried out in an ethereal solvent such as diethyl ether or tetrahydrofuran. Some examples of this chemistry can be found in Miwa et al., Synlett 1994, 107-108 and Kao et al., Journal of Organic Chemistry 2002, 67, 6772-6787.

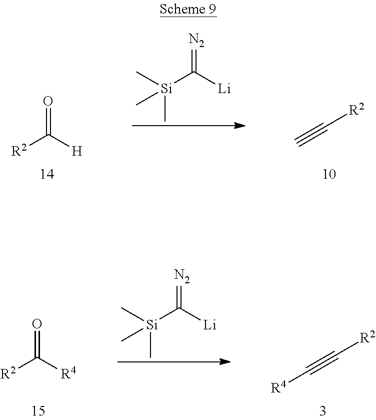

Transformation of compounds of Formula 1a to compounds of Formula 1c (containing a thiocarbonyl group) may be carried out with phosphorous pentasulfide as shown in Scheme 10. Likewise, transformation of compounds of Formula 1b to compounds of Formula 1d (containing a thiocarbonyl group) may be carried out with phosphorous pentasulfide. For conditions to carry out this reaction, see Journal of Heterocyclic Chemistry 2007, 44, 1109-1114, Saudi Pharmaceutical Journal 2008, 16, 33-42, Flavour and Fragrance Journal 2006, 21, 175-184 and Archiv der Pharmazie 1984, 317, 938-45.

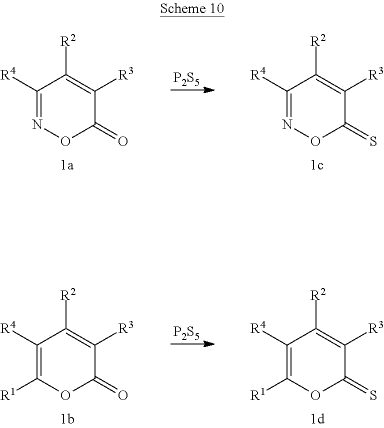

Syntheses and transformations of pyrones as well as methods to introduce functional groups onto this ring system have been recently reviewed by Ram and Srivastava in Current Organic Chemistry 2001, 5, 571-599.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “m” means multiplet.

### Example 1

### Preparation of 4-(3,5-dimethoxyphenyl)-3-(2-fluorophenyl)-5-(2,4,6-trifluorophenyl)-6H-1,2-oxazin-6-one

### Step A: Preparation of 4-(3,5-dimethoxyphenyl)-3-(2-fluorophenyl)-5-(2,4,6-trifluorophenyl)-6H-1,2-oxazin-6-one

To a mixture of 4-(3,5-dimethoxyphenyl)-5-(2-fluorophenyl)-5-hydroxy-3-(2,4,6-trifluorophenyl)-2(3H)-furanone (1.4 g, 3.0 mmol, prepared as described in Patent Application Publication WO 2010/036553) in ethanol (6 mL) was added hydroxylamine (50% in water, 1.2 mL) and solid sodium hydroxide (200 mg, 5 mmol). The reaction mixture was heated at reflux for 23 h. The reaction mixture was poured into ice water (50 mL) and acidified with concentrated hydrochloric acid. The solid that formed was filtered, washed with water and allowed to air dry for 1 h. The solid was mixed with glacial acetic acid (6 mL) and heated at reflux for 2 h. The reaction mixture was concentrated under reduced pressure and the resulting solid was collected by filtration and washed with water. The solid was dissolved in dichloromethane (30 mL), dried over magnesium sulfate, filtered and concentrated under reduced pressure. The resulting material was purified by column chromatography on 20 g of silica gel (15:1 dichloromethane/ethyl acetate as eluant) to provide the title compound, a compound of the invention, as a solid (0.3 g).

¹H NMR (CDCl₃) δ 3.52 (s, 6H), 6.00 (m, 2H), 6.25 (m, 1H), 6.65 (m, 1H), 6.92 (m, 2H), 7.17 (m, 1H), 7.36 (m, 2H).

### Example 2

### Preparation of 5-bromo-4-(2,4-difluorophenyl)-3,6-dimethyl-2H-pyran-2-one

### Step A: Preparation of 4-(2,4-difluorophenyl)-3,6-dimethyl-2H-pyran-2-one

To a mixture solution of ethyl 2-methylacetoacetate (1.25 g, 8.68 mmol) and 2,4-difluorophenylacetylene (1.3 g, 6.1 mmol) in toluene (10 mL) was added 4 Å molecular sieves (400 mg) and ReBr(CO)₃(thf)₂ (180 mg, 0.21 mmol). The reaction mixture was heated at 90° C. for 60 h, after which time tetrabutylammonium fluoride (0.5 M in tetrahydrofuran, 0.5 mL) was added and stirring was continued at 23° C. for 18 h. The reaction mixture was concentrated onto silica gel and then purified by column chromatography (10:1 hexanes/ethyl acetate as eluant) to provide the title compound as a solid (1.3 g).

¹H NMR (CDCl₃) δ 1.95 (s, 3H), 2.26 (s, 3H), 5.89 (s, 1H), 6.97 (m, 2H), 7.22 (m, 1H).

### Step B: Preparation of 5-bromo-4-(2,4-difluorophenyl)-3,6-dimethyl-2H-pyran-2-one

To a mixture of 4-(2,4-difluorophenyl)-3,6-dimethyl-2H-pyran-2-one (i.e. the product from Step A) (1.1 g, 4.7 mmol) in N,N-dimethylformamide (15 mL) was added N-bromosuccinimide (900 mg, 5.0 mmol). The reaction mixture was heated at 80° C. for 14 h, and pour into to ice water (80 mL). The resulting mixture was extracted with diethyl ether (50 mL) and the organic layer was washed with water (40 mL), dried over magnesium sulfate, filtered and concentrated under reduce pressure. The resulting material was purified by column chromatography on 20 g of silica gel (10:1 hexanes/ethyl acetate as eluant) to provide the title compound, a compound of the invention, as an oil (1.0 g).

¹H NMR (CDCl₃) δ 1.91 (s, 3H), 2.46 (s, 3H), 6.95 (m, 1H), 7.00 (m, 1H), 7.09 (m, 1H).

### Example 3

### Preparation of 4-(2,4-difluorophenyl)-5-(3,5-dimethoxyphenyl)-3,6-dimethyl-2H-pyran-2-one

### Step A: Preparation of 4-(2,4-difluorophenyl)-5-(3,5-dimethoxyphenyl)-3,6-dimethyl-2H-pyran-2-one

A mixture of 5-bromo-4-(2,4-difluorophenyl)-3,6-dimethyl-2H-pyran-2-one (i.e. the product of Example 2, step B) (300 mg, 0.95 mmol), 3,5-dimethoxyphenylboronic acid (270 mg, 1.5 mmol), potassium phosphate tribasic (700 mg, 3.3 mmol), palladium acetate (9 mg, 0.4 mmol) and 2-dicyclohexylphosphino-2′,6′-dimethoxy-1,1′-biphenyl (30 mg, 0.073 mmol) in toluene (5 mL) was heated at 100° C. for 18 h. After 18 h, a saturated aqueous solution of ethylenediaminetetraacetic acid disodium salt (50 mL) and dichloromethane (50 mL) was added to the reaction mixture, the layers were separated and the dichloromethane layer was dried over magnesium sulfate, filtered and concentrated under reduced pressure. The resulting material was purified by column chromatography on 20 g of silica gel (5:1 hexanes/ethyl acetate as eluant) to provide a solid. The solid was triturated with hexanes to provide the title compound, a compound of the present invention, as a cream colored solid (300 mg).

¹H NMR (CDCl₃) δ 1.89 (s, 3H), 2.15 (s, 3H), 3.65 (s, 3H), 3.69 (s, 3H), 6.22 (m, 2H), 6.26 (m, 1H), 6.73 (m, 2H), 6.84 (m, 1H).

### Example 4

### Preparation of 4-(3,5-dimethoxyphenyl)-3,6-dimethyl-5-(2,4,6-trifluorophenyl)-2H-pyran-2-one

### Step A: Preparation of (3,5-dimethoxyphenyl)(2,4,6-trifluorophenyl)methanone

To a mixture of 1,3,5-trifluorobenzene (4.77 g, 36 mmol) cooled to −78° C. and was added dropwise with n-butyllithium (2.5 N in hexanes, 15 mL, 36 mmol). The reaction mixture was stirred for 10 minutes, and then 3,5-dimethoxybenzaldehyde (5.0 g, 30 mmol) dissolved in tetrahydrofuran (20 mL) was added and mixture was allowed to warm to 23° C. After stirring at this temperature for 18 h, saturated aqueous ammonium chloride solution (60 mL) was added. The reaction mixture was extracted with ethyl acetate (3×30 mL) and the combined organic extracts were dried over magnesium sulfate, filtered and concentrated under reduced pressure. The resulting material was purified by column chromatography on 80 g of silica gel (0 to 60% gradient of ethyl acetate in hexanes as eluant) to provide an oil. The oil was dissolved in tetrahydrofuran (300 mL) and treated with activated manganese dioxide (23 g, 256 mmol). The mixture was stirred at 25° C. for 18 h and then filtered over Celite® (diatomaceous filter aid) and concentrated under reduced pressure. The resulting material was purified by column chromatography on 80 g of silica gel (0 to 50% gradient of ethyl acetate in hexanes as eluant) to provide the title compound as an oil (3.0 g) which solidified on standing.

¹H NMR (CDCl₃) δ 3.83 (s, 6H), 6.69-6.82 (m, 3H), 6.98 (d, 2H).

### Step B: Preparation of 2-[2-(3,5-dimethyoxyphenyl)ethynyl]-1,3,5-trifluorobenzene

A mixture of trimethylsilyldiazomethane (2.0 M in hexanes, 5.5 mL, 11 mmol) in tetrahydrofuran (50 mL) was cooled to −78° C. and then n-butyllithium (2.5 N in hexanes, 4.5 mL, 11 mmol) was added. The reaction mixture was stirred at this temperature for 30 minutes, and then (3,5-dimethoxyphenyl)(2,4,6-trifluorophenyl)methanone (i.e. the product of Step A) (3.0 g, 10 mmol) in tetrahydrofuran (10 mL) was added and the mixture was allowed to warm to 25° C. and stirred for 18 h. The reaction mixture was concentrated under reduced pressure. The resulting material was purified by chromatography on 80 g of silica gel (0 to 100% gradient of ethyl acetate in hexanes as eluant) to provide the title compound as a yellow solid (2.12 g).

¹H NMR (CDCl₃) δ 3.81 (s, 6H), 6.47-6.52 (m, 1H), 6.67-6.76 (m, 4H).

### Step C: Preparation of 4-(3,5-dimethoxyphenyl)-3,6-dimethyl-5-(2,4,6-trifluorophenyl)-2H-pyran-2-one and 5-(3,5-dimethoxyphenyl)-3,6-dimethyl-4-(2,4,6-trifluorophenyl)-2H-pyran-2-one

To a mixture of 2-[2-(3,5-dimethyoxyphenyl)ethynyl]-1,3,5-trifluorobenzene (i.e. the product of Step B) (1.0 g, 3.4 mmol) in toluene (10 mL) was added ethyl 2-methylacetoacetate (0.48 g, 3.4 mmol), dibromohexacarbonylbis(tetrahydrofuran)-dirhenium (58 mg, 0.069 mmol) and 4 Å molecular sieves (powered, 116 mg). The reaction mixture was heated at reflux for 2 days. The reaction mixture was concentrated under reduced pressure and the resulting material was purified by column chromatography on 40 g of silica gel eluting (0 to 100% gradient of ethyl acetate in hexanes as eluant) to provide the title compounds, compounds of the present invention, as a solid (0.77 g, 60:40 mixture).

¹H NMR (CDCl₃) δ 1.91 (s, 1.2H), 1.94 (s, 1.8H), 2.09 (s, 1.8H), 2.14 (s, 1.2H), 3.69 (s, 6H), 6.13 (m, 1.2H), 6.18 (m, 0.8H), 6.30 (m, 1H), 6.55 (m, 2H).

### Step D: Preparation of 4-(3,5-dimethoxyphenyl)-3,6-dimethyl-5-(2,4,6-trifluorophenyl)-2H-pyran-2-one

To a mixture of 4-(3,5-dimethoxyphenyl)-3,6-dimethyl-5-(2,4,6-trifluorophenyl)-2H-pyran-2-one and 5-(3,5-dimethoxyphenyl)-3,6-dimethyl-4-(2,4,6-trifluorophenyl)-2H-pyran-2-one (i.e. the product of Step C) (0.70 g, 1.8 mmol) in N,N-dimethylformamide (10 mL) was added N-chlorosuccinimide (0.12 g, 0.89 mmol). The reaction mixture was heated at 65° C. for 3 h, poured into water (50 mL) and extracted with ethyl acetate (3×30 mL). The combined organic layers were dried over magnesium sulfate, filtered and concentrated under reduced pressure. The resulting material was purified by column chromatography on 40 g of silica gel eluting (0 to 100% gradient of ethyl acetate in hexanes as eluant) to provide the title compound, a compound of the present invention, as a white solid (0.37 g).

¹H NMR (CDCl₃) δ 1.93 (s, 3H), 2.09 (s, 3H), 3.69 (s, 6H), 6.13 (d, 2H), 6.28-6.30 (m, 1H), 6.52-6.56 (m, 2H).

### Example 5

### Preparation of 4-(2-chloro-3,5-dimethoxyphenyl)-3,6-dimethyl-5-(2,4,6-trifluorophenyl)-2H-pyran-2-one and 5-(2-chloro-3,5-dimethoxyphenyl)-3,6-dimethyl-4-(2,4,6-trifluorophenyl)-2H-pyran-2-one

### Step A: Preparation of 4-(2-chloro-3,5-dimethoxyphenyl)-3,6-dimethyl-5-(2,4,6-trifluorophenyl)-2H-pyran-2-one and 5-(2-chloro-3,5-dimethoxyphenyl)-3,6-dimethyl-4-(2,4,6-trifluorophenyl)-2H-pyran-2-one

Continued gradient elution of the column from Example 4, Step D gives the title compounds as a 50:50 mixture of isomers, a mixture of the present invention (0.22 g).

¹H NMR (CDCl₃) δ 1.87 (s, 1.5H), 1.92 (s, 1.5H), 2.08 (s, 1.5H), 2.11 (s, 1.5H), 3.71 (m, 3H), 3.82 (s, 3H), 6.18 (m, 0.5H), 6.26 (m, 0.5H), 6.37 (m, 1H), 6.56 (m, 2H).

### Example 6

### Preparation of 4-(2-chloro-3,5-dimethoxyphenyl)-3,6-dimethyl-5-(2,4,6-trifluorophenyl)-2H-pyran-2-one

### Step A: Preparation of 4-(2-chloro-3,5-dimethoxyphenyl)-3,6-dimethyl-5-(2,4,6-trifluorophenyl)-2H-pyran-2-one

To a mixture of 4-(3,5-dimethoxyphenyl)-3,6-dimethyl-5-(2,4,6-trifluorophenyl)-2H-pyran-2-one (i.e. the product of Example 4, Step D) (0.150 g, 0.384 mmol) was in N,N-dimethylformamide (10 mL) and added N-chlorosuccinimide (0.051 g, 0.384 mmol). The reaction mixture was heated at 60° C. for 3 h, and then poured into water (50 mL) and extracted with ethyl acetate (3×30 mL). The combined organic layers were dried over magnesium sulfate, filtered and concentrated under reduce pressure. The resulting material was purified by column chromatography on 12 g of silica gel eluting (0 to 60% gradient of ethyl acetate in hexanes as eluant) to provide the title compound, a compound of the present invention, as a white solid (0.080 g).

¹H NMR (CDCl₃) δ 1.87 (s, 3H), 2.11 (s, 3H), 3.71 (s, 3H), 3.81 (s, 3H), 6.13-6.20 (m, 1H), 6.37 (d, 1H), 6.48-6.61 (m, 2H).

By the procedures described herein together with methods known in the art, the following compounds of Tables 1 to 58C can be prepared. The following abbreviations are used in the Tables which follow: s means secondary, n means normal, i means iso, c means cyclo, Me means methyl, Et means ethyl, Pr means propyl, i-Pr means isopropyl, Bu means butyl, Ph means phenyl, OMe means methoxy, OEt means ethoxy, SMe means methylthio, SEt means ethylthio, Ph means phenyl and Py means pyridinyl.

TABLE 1

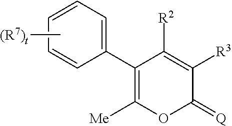

R² is 3,5-di-MeO—Ph; (R⁷)ₜ is 2-F; and Q is O.
R³
MeCO₂
EtCO₂
n-Pr—CO₂
MeC(═O)
MeOCH₂
F₃C
Me
Et
i-Pr
c-Pr
s-Bu
ClCH₂
c-pentyl
c-hexyl
MeC(═CH₂)
n-Pr
Me₂C(OH)
NCCH₂
Ph
2-F—Ph
2-Cl—Ph
3-F—Ph
4-F—Ph
2-Me—Ph
4-Cl—Ph
2-MeO—Ph
2-CF₃—Ph
2-CN—Ph
2-Br—Ph
c-Bu
3-F-2-Py
5-Cl-2-Py
2-F-3-Py
thiazol-2-yl
2-Pyl

The present disclosure also includes Tables 1A through 71A, each of which is constructed the same as Table 1 above except that the row heading in Table 1 (i.e. “R2 is 3,5-di-MeO-Ph; (R7)t is 2-F; and Q is O”) is replaced with the respective row heading shown below. Thus, for example, in Table 1A the row heading is “R2 is 2-Cl, 5-MeO-Ph; (R7)t is 2-F; and Q is O”, and R3 is as defined in Table 1 above. Tables 2A through 71A are constructed similarly.

| Table | Row Heading |
|---|---|
| 1A | R² is 2-Cl, 5-MeO—Ph; (R⁷)ₜ is 2-F; and Q is O. |
| 2A | R² is 3,5-di-MeO—Ph; (R⁷)ₜ is 4-F; and Q is O. |
| 3A | R² is 2-Cl, 5-MeO—Ph; (R⁷)ₜ is 4-F; and Q is O. |
| 4A | R² is 3,5-di-MeO—Ph; (R⁷)ₜ is 2,4-di-F; and Q is O. |
| 5A | R² is 2-Cl, 5-MeO—Ph; (R⁷)ₜ is 2,4-di-F; and Q is O. |
| 6A | R² is 3,5-di-MeO—Ph; (R⁷)ₜ is 2,6-di-F; and Q is O. |
| 7A | R² is 2-Cl, 5-MeO—Ph; (R⁷)ₜ is 2,6-di-F; and Q is O. |
| 8A | R² is 3,5-di-MeO—Ph; (R⁷)ₜ is 2,4,6-tri-F; and Q is O. |
| 9A | R² is 2-Cl, 5-MeO—Ph; (R⁷)ₜ is 2,4,6-tri-F; and Q is O. |
| 10A | R² is 3,5-di-MeO—Ph; (R⁷)ₜ is 2-Cl; and Q is O. |
| 11A | R² is 2-Cl, 5-MeO—Ph; (R⁷)ₜ is 2-Cl; and Q is O. |
| 12A | R² is 3,5-di-MeO—Ph; (R⁷)ₜ is 2-Cl, 4-F; and Q is O. |
| 13A | R² is 2-Cl, 5-MeO—Ph; (R⁷)ₜ is 2-Cl, 4-F; and Q is O. |
| 14A | R² is 3,5-di-MeO—Ph; (R⁷)ₜ is 2,3,6-tri-F; and Q is O. |
| 15A | R² is 2-Cl, 5-MeO—Ph; (R⁷)ₜ is 2,3,6-tri-F; and Q is O. |
| 16A | R² is 3,5-di-MeO—Ph; (R⁷)ₜ is 2,6-di-F, 4-MeO; and Q is O. |
| 17A | R² is 2-Cl, 5-MeO—Ph; (R⁷)ₜ is 2,6-di-F, 4-MeO; and Q is O. |
| 18A | R² is 3,5-di-MeO—Ph; (R⁷)ₜ is 2,6-di-F, 4-MeNH(CH₂)₃O; |
| | and Q is O. |
| 19A | R² is 2-Cl, 5-MeO—Ph; (R⁷)ₜ is 2,6-di-F, 4-MeNH(CH₂)₃O; |
| | and Q is O. |
| 20A | R² is 3,5-di-MeO—Ph; (R⁷)ₜ is 2,6-di-F, 3-MeO; and Q is O. |
| 21A | R² is 2-Cl, 5-MeO—Ph; (R⁷)ₜ is 2,6-di-F, 3-MeO; and Q is O. |
| 22A | R² is 2-Cl, 3,5-di-MeO—Ph; (R⁷)ₜ is 2-F; and Q is O. |
| 23A | R² is 2-Cl, 3,5-MeO—Ph; (R⁷)ₜ is 4-F; and Q is O. |
| 24A | R² is 2-Cl, 3,5-MeO—Ph; (R⁷)ₜ is 2,4-di-F; and Q is O. |
| 25A | R² is 2-Cl, 3,5-di-MeO—Ph; (R⁷)ₜ is 2,6-di-F; and Q is O. |
| 26A | R² is 2-Cl, 3,5-di-MeO—Ph; (R⁷)ₜ is 2,4,6-tri-F; and Q is O. |
| 27A | R² is 2-Cl, 3,5-di-MeO—Ph; (R⁷)ₜ is 4-Cl; and Q is O. |
| 28A | R² is 2-Cl, 3,5-di-MeO—Ph; (R⁷)ₜ is 4-MeO; and Q is O. |
| 29A | R² is 2-Cl, 3,5-di-MeO—Ph; (R⁷)ₜ is 2,3,6-tri-F; and Q is O. |
| 30A | R² is 2-Cl, 3,5-di-MeO—Ph; (R⁷)ₜ is 2,6-di-F, 4-MeO; |
| | and Q is O. |
| 31A | R² is 2-Cl, 3,5-di-MeO—Ph; (R⁷)ₜ is 2,6-di-F, 4-MeNH(CH₂)₃O; |
| | and Q is O. |
| 32A | R² is 2-Cl, 3,5-di-MeO—Ph; (R⁷)ₜ is 2,6-di-F, 3-MeO; |
| | and Q is O. |
| 33A | R² is 3,5-di-MeO—Ph; (R⁷)ₜ is 2-Cl, 6-F; and Q is O. |
| 34A | R² is 2-Cl, 5-MeO—Ph; (R⁷)ₜ is 2-Cl, 6-F; and Q is O. |
| 35A | R² is 2-Cl, 3-MeO—Ph; (R⁷)ₜ is 2-F; and Q is O. |
| 36A | R² is 2-Cl, 3-MeO—Ph; (R⁷)ₜ is 4-F; and Q is O. |
| 37A | R² is 2-Cl, 3-MeO—Ph; (R⁷)ₜ is 2,4-di-F; and Q is O. |
| 38A | R² is 2-Cl, 3-MeO—Ph; (R⁷)ₜ is 2,6-di-F; and Q is O. |
| 39A | R² is 2-Cl, 3-MeO—Ph; (R⁷)ₜ is 2,4,6-tri-F; and Q is O. |
| 40A | R² is 2-Cl, 3-MeO—Ph; (R⁷)ₜ is 2,3,6-tri-F; and Q is O. |
| 41A | R² is 2-Cl, 3-MeO—Ph; (R⁷)ₜ is 2,6-di-F, 4-OMe; and Q is O. |
| 42A | R² is 2-Cl, 3-MeO—Ph; (R⁷)ₜ is 2-Cl,6-F; and Q is O. |
| 43A | R² is 2-Cl, 3-MeO—Ph; (R⁷)ₜ is 2-Cl,4-F; and Q is O. |
| 44A | R² is 2-Cl, 3-MeO—Ph; (R⁷)ₜ is 2-F; and Q is O. |
| 45A | R² is 2-Cl—Ph; (R⁷)ₜ is 4-F; and Q is O. |
| 46A | R² is 2-Cl—Ph; (R⁷)ₜ is 2,4-di-F; and Q is O. |
| 47A | R² is 2-Cl—Ph; (R⁷)ₜ is 2,6-di-F; and Q is O. |
| 48A | R² is 2-Cl—Ph; (R⁷)ₜ is 2,4,6-tri-F; and Q is O. |
| 49A | R² is 2-Cl—Ph; (R⁷)ₜ is 2,3,6-tri-F; and Q is O. |
| 50A | R² is 2-Cl—Ph; (R⁷)ₜ is 2,6-di-F, 4-OMe; and Q is O. |
| 51A | R² is 2-Cl—Ph; (R⁷)ₜ is 2-Cl,6-F; and Q is O. |
| 52A | R² is 2-Cl—Ph; (R⁷)ₜ is 2-Cl,4-F; and Q is O. |
| 53A | R² is 3,5-di-MeO—Ph; (R⁷)ₜ is 2,4-di-F; and Q is S. |
| 54A | R² is 2-Cl, 5-MeO—Ph; (R⁷)ₜ is 2,4-di-F; and Q is S. |
| 55A | R² is 3,5-di-MeO—Ph; (R⁷)ₜ is 2,6-di-F; and Q is S. |
| 56A | R² is 2-Cl, 5-MeO—Ph; (R⁷)ₜ is 2,6-di-F; and Q is S. |
| 57A | R² is 3,5-di-MeO—Ph; (R⁷)ₜ is 2,4,6-tri-F; and Q is S. |
| 58A | R² is 2-Cl, 5-MeO—Ph; (R⁷)ₜ is 2,4,6-tri-F; and Q is S. |
| 59A | R² is 3,5-di-MeO—Ph; (R⁷)ₜ is 2,3,6-tri-F; and Q is S. |
| 60A | R² is 2-Cl, 5-MeO—Ph; (R⁷)ₜ is 2,3,6-tri-F; and Q is S. |
| 61A | R² is 3,5-di-MeO—Ph; (R⁷)ₜ is 2,6-di-F, 4-MeO; and Q is S. |
| 62A | R² is 2-Cl, 5-MeO—Ph; (R⁷)ₜ is 2,6-di-F, 4-MeO; and Q is S. |
| 63A | R² is 3,5-di-MeO—Ph; (R⁷)ₜ is 2,6-di-F, 4-MeNH(CH₂)₃O; |
| | and Q is S. |
| 64A | R² is 2-Cl, 5-Me—Ph; (R⁷)ₜ is 2,6-di-F, 4-MeNH(CH₂)₃O; |
| | and Q is S. |
| 65A | R² is 3,5-di-MeO—Ph; (R⁷)ₜ is 2,6-di-F, 3-MeO; and Q is S. |
| 66A | R² is 2-Cl, 5-Me—Ph; (R⁷)ₜ is 2,6-di-F, 3-MeO; and Q is S. |
| 67A | R² is 2-Cl, 3,5-MeO—Ph; (R⁷)ₜ is 2,4-di-F; and Q is S. |
| 68A | R² is 2-Cl, 3,5-di-MeO—Ph; (R⁷)ₜ is 2,6-di-F; and Q is S. |
| 69A | R² is 2-Cl, 3,5-di-MeO—Ph; (R⁷)ₜ is 2,4,6-tri-F; and Q is S. |
| 70A | R² is 2-Cl, 3,5-di-MeO—Ph; (R⁷)ₜ is 2,6-di-F; and Q is S. |
| 71A | R² is 2-Cl, 3,5-di-MeO—Ph; (R⁷)ₜ is 2,4,6-tri-F; and Q is S. |

TABLE 2

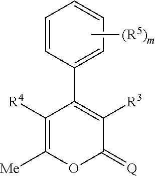

R⁴ is 3,5-di-MeO—Ph; (R⁵)ₘ is 2-F; and Q is O.
R³
MeCO₂
EtCO₂
n-Pr—CO₂
MeC(═O)
MeOCH₂
F₃C
Me
Et
i-Pr
c-Pr
s-Bu
ClCH₂
c-pentyl
c-hexyl
MeC(═CH₂)
n-Pr
Me₂C(OH)
NCCH₂
Ph
2-F—Ph
2-Cl—Ph
3-F—Ph
4-F—Ph
2-Me—Ph
4-Cl—Ph
2-MeO—Ph
2-CF₃—Ph
2-CN—Ph
2-Br—Ph
c-Bu
3-F-2-Py
5-Cl-2-Py
2-F-3-Py
thiazol-2-yl
2-Py

The present disclosure also includes Tables 1B through 58B, each of which is constructed the same as Table 2 above except that the row heading in Table 2 (i.e. “R4 is 3,5-di-MeO-Ph; (R5)m is 2-F; and Q is O”) is replaced with the respective row heading shown below. Thus, for example, in Table 1B the row heading is “R4 is 2-Cl, 5-MeO-Ph; (R5)m is 2-F; and Q is O”, and R3 is as defined in Table 2 above. Tables 2B through 58B are constructed similarly.

| Table | Row Heading |
|---|---|
| 1B | R⁴ is 2-Cl, 5-MeO—Ph; (R⁵)ₘ is 2-F; and Q is O. |
| 2B | R⁴ is 3,5-di-MeO—Ph; (R⁵)ₘ is 4-F; and Q is O. |
| 3B | R⁴ is 2-Cl, 5-MeO—Ph; (R⁵)ₘ is 4-F; and Q is O. |
| 4B | R⁴ is 3,5-di-MeO—Ph; (R⁵)ₘ is 2,4-di-F; and Q is O. |
| 5B | R⁴ is 2-Cl, 5-MeO—Ph; (R⁵)ₘ is 2,4-di-F; and Q is O. |
| 6B | R⁴ is 3,5-di-MeO—Ph; (R⁵)ₘ is 2,6-di-F; and Q is O. |
| 7B | R⁴ is 2-Cl, 5-MeO—Ph; (R⁵)ₘ is 2,6-di-F; and Q is O. |
| 8B | R⁴ is 3,5-di-MeO—Ph; (R⁵)ₘ is 2,4,6-tri-F; and Q is O. |
| 9B | R⁴ is 2-Cl, 5-MeO—Ph; (R⁵)ₘ is 2,4,6-tri-F; and Q is O. |
| 10B | R⁴ is 3,5-di-MeO—Ph; (R⁵)ₘ is 2-Cl; and Q is O. |
| 11B | R⁴ is 2-Cl, 5-MeO—Ph; (R⁵)ₘ is 2-Cl; and Q is O. |
| 12B | R⁴ is 3,5-di-MeO—Ph; (R⁵)ₘ is 2-Cl, 4-F; and Q is O. |
| 13B | R⁴ is 2-Cl, 5-MeO—Ph; (R⁵)ₘ is 2-Cl, 4-F; and Q is O. |
| 14B | R⁴ is 3,5-di-MeO—Ph; (R⁵)ₘ is 2,3,6-tri-F; and Q is O. |
| 15B | R⁴ is 2-Cl, 5-MeO—Ph; (R⁵)ₘ is 2,3,6-tri-F; and Q is O. |
| 16B | R⁴ is 3,5-di-MeO—Ph; (R⁵)ₘ is 2,6-di-F, 4-MeO; and Q is O. |
| 17B | R⁴ is 2-Cl, 5-MeO—Ph; (R⁵)ₘ is 2,6-di-F, 4-MeO; and Q is O. |
| 18B | R⁴ is 3,5-di-MeO—Ph; (R⁵)ₘ is 2,6-di-F, 4-MeNH(CH₂)₃O; |
| | and Q is O. |
| 19B | R⁴ is 2-Cl, 5-MeO—Ph; (R⁵)ₘ is 2,6-di-F, 4-MeNH(CH₂)₃O; |
| | and Q is O. |
| 20B | R⁴ is 3,5-di-MeO—Ph; (R⁵)ₘ is 2,6-di-F, 3-MeO; and Q is O. |
| 21B | R⁴ is 2-Cl, 5-MeO—Ph; (R⁵)ₘ is 2,6-di-F, 3-MeO; and Q is O. |
| 22B | R⁴ is 2-Cl, 3,5-di-MeO—Ph; (R⁵)ₘ is 2-F; and Q is O. |
| 23B | R⁴ is 2-Cl, 3,5-MeO—Ph; (R⁵)ₘ is 4-F; and Q is O. |
| 24B | R⁴ is 2-Cl, 3,5-MeO—Ph; (R⁵)ₘ is 2,4-di-F; and Q is O. |
| 25B | R⁴ is 2-Cl, 3,5-di-MeO—Ph; (R⁵)ₘ is 2,6-di-F; and Q is O. |
| 26B | R⁴ is 2-Cl, 3,5-di-MeO—Ph; (R⁵)ₘ is 2,4,6-tri-F; and Q is O. |
| 27B | R⁴ is 2-Cl, 3,5-di-MeO—Ph; (R⁵)ₘ is 4-Cl; and Q is O. |
| 28B | R⁴ is 2-Cl, 3,5-di-MeO—Ph; (R⁵)ₘ is 4-MeO; and Q is O. |
| 29B | R⁴ is 2-Cl, 3,5-di-MeO—Ph; (R⁵)ₘ is 2,3,6-tri-F; and Q is O. |
| 30B | R⁴ is 2-Cl, 3,5-di-MeO—Ph; (R⁵)ₘ is 2,6-di-F, 4-MeO; |
| | and Q is O. |
| 31B | R⁴ is 2-Cl, 3,5-di-MeO—Ph; (R⁵)ₘ is 2,6-di-F, |
| | 4-MeNH(CH₂)₃O; and Q is O. |
| 32B | R⁴ is 2-Cl, 3,5-di-MeO—Ph; (R⁵)ₘ is 2,6-di-F, 3-MeO; |
| | and Q is O. |
| 33B | R⁴ is 3,5-di-MeO—Ph; (R⁵)ₘ is 2-Cl, 6-F; and Q is O. |
| 34B | R⁴ is 2-Cl, 5-MeO—Ph; (R⁵)ₘ is 2-Cl, 6-F; and Q is O. |
| 35B | R⁴ is 2-Cl, 3-MeO—Ph; (R⁵)ₘ is 2-F; and Q is O. |
| 36B | R⁴ is 2-Cl, 3-MeO—Ph; (R⁵)ₘ is 4-F; and Q is O. |
| 37B | R⁴ is 2-Cl, 3-MeO—Ph; (R⁵)ₘ is 2,4-di-F; and Q is O. |
| 38B | R⁴ is 2-Cl, 3-MeO—Ph; (R⁵)ₘ is 2,6-di-F; and Q is O. |
| 39B | R⁴ is 2-Cl, 3-MeO—Ph; (R⁵)ₘ is 2,4,6-tri-F; and Q is O. |
| 40B | R⁴ is 2-Cl, 3-MeO—Ph; (R⁵)ₘ is 2,3,6-tri-F; and Q is O. |
| 41B | R⁴ is 2-Cl, 3-MeO—Ph; (R⁵)ₘ is 2,6-di-F, 4-OMe; and Q is O. |
| 42B | R⁴ is 2-Cl, 3-MeO—Ph; (R⁵)ₘ is 2-Cl,6-F; and Q is O. |
| 43B | R⁴ is 2-Cl, 3-MeO—Ph; (R⁵)ₘ is 2-Cl,4-F; and Q is O. |
| 44B | R⁴ is 2-Cl, 3-MeO—Ph; (R⁵)ₘ is 2-F; and Q is O. |
| 45B | R⁴ is 2-Cl—Ph; (R⁵)ₘ is 4-F; and Q is O. |
| 46B | R⁴ is 2-Cl—Ph; (R⁵)ₘ is 2,4-di-F; and Q is O. |
| 47B | R⁴ is 2-Cl—Ph; (R⁵)ₘ is 2,6-di-F; and Q is O. |
| 48B | R⁴ is 2-Cl—Ph; (R⁵)ₘ is 2,4,6-tri-F; and Q is O. |
| 49B | R⁴ is 2-Cl—Ph; (R⁵)ₘ is 2,3,6-tri-F; and Q is O. |
| 506 | R⁴ is 2-Cl—Ph; (R⁵)ₘ is 2,6-di-F, 4-OMe; and Q is O. |
| 51B | R⁴ is 2-Cl—Ph; (R⁵)ₘ is 2-Cl,6-F; and Q is O. |
| 52B | R⁴ is 2-Cl—Ph; (R⁵)ₘ is 2-Cl,4-F; and Q is O. |
| 53B | R⁴ is 3,5-di-MeO—Ph; (R⁵)ₘ is 2,4-di-F; and Q is S. |
| 54B | R⁴ is 2-Cl, 5-MeO—Ph; (R⁵)ₘ is 2,4-di-F; and Q is S. |
| 55B | R⁴ is 3,5-di-MeO—Ph; (R⁵)ₘ is 2,6-di-F; and Q is S. |
| 56B | R⁴ is 2-Cl, 5-MeO—Ph; (R⁵)ₘ is 2,6-di-F; and Q is S. |
| 57B | R⁴ is 3,5-di-MeO—Ph; (R⁵)ₘ is 2,4,6-tri-F; and Q is S. |
| 58B | R⁴ is 2-Cl, 5-MeO—Ph; (R⁵)ₘ is 2,4,6-tri-F; and Q is S. |

TABLE 3

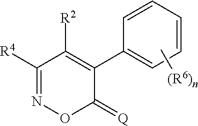

R² is 3,5-di-MeO—Ph (R⁶)ₙ is 2-F; and Q is O.
R⁴
MeCO₂
EtCO₂
n-Pr—CO₂
MeC(═O)
MeOCH₂
F₃C
Me
Et
i-Pr
c-Pr
s-Bu
ClCH₂
c-pentyl
c-hexyl
MeC(═CH₂)
n-Pr
Me₂C(OH)
NCCH₂
Ph
2-F—Ph
2-Cl—Ph
3-F—Ph
4-F—Ph
2-Me—Ph
4-Cl—Ph
2-MeO—Ph
2-CF₃—Ph
2-CN—Ph
2-Br—Ph
c-Bu
3-F-2-Py
5-Cl-2-Py
2-F-3-Py
thiazol-2-yl
2-Py

The present disclosure also includes Tables 1C through 58C, each of which is constructed the same as Table 3 above except that the row heading in Table 1 (i.e. “R2 is 3,5-di-MeO-Ph; (R6)n is 2-F; and Q is O”) is replaced with the respective row heading shown below. Thus, for example, in Table 1C the row heading is “R2 is 2-Cl, 5-MeO-Ph; (R6)n is 2-F; and Q is O”, and R4 is as defined in Table 3 above. Tables 2C through 58C are constructed similarly.

| Table | Row Heading |
|---|---|
| 1C | R² is 2-Cl, 5-MeO—Ph; (R⁶)ₙ is 2-F; and Q is O. |
| 2C | R² is 3,5-di-MeO—Ph; (R⁶)ₙ is 4-F; and Q is O. |
| 3C | R² is 2-Cl, 5-MeO—Ph; (R⁶)ₙ is 4-F; and Q is O. |
| 4C | R² is 3,5-di-MeO—Ph; (R⁶)ₙ is 2,4-di-F; and Q is O. |
| 5C | R² is 2-Cl, 5-MeO—Ph; (R⁶)ₙ is 2,4-di-F; and Q is O. |
| 6C | R² is 3,5-di-MeO—Ph; (R⁶)ₙ is 2,6-di-F; and Q is O. |
| 7C | R² is 2-Cl, 5-MeO—Ph; (R⁶)ₙ is 2,6-di-F; and Q is O. |
| 8C | R² is 3,5-di-MeO—Ph; (R⁶)ₙ is 2,4,6-tri-F; and Q is O. |
| 9C | R² is 2-Cl, 5-MeO—Ph; (R⁶)ₙ is 2,4,6-tri-F; and Q is O. |
| 10C | R² is 3,5-di-MeO—Ph; (R⁶)ₙ is 2-Cl; and Q is O. |
| 11C | R² is 2-Cl, 5-MeO—Ph; (R⁶)ₙ is 2-Cl; and Q is O. |
| 12C | R² is 3,5-di-MeO—Ph; (R⁶)ₙ is 2-Cl, 4-F; and Q is O. |
| 13C | R² is 2-Cl, 5-MeO—Ph; (R⁶)ₙ is 2-Cl, 4-F; and Q is O. |
| 14C | R² is 3,5-di-MeO—Ph; (R⁶)ₙ is 2,3,6-tri-F; and Q is O. |
| 15C | R² is 2-Cl, 5-MeO—Ph; (R⁶)ₙ is 2,3,6-tri-F; and Q is O. |
| 16C | R² is 3,5-di-MeO—Ph; (R⁶)ₙ is 2,6-di-F, 4-MeO; and Q is O. |
| 17C | R² is 2-Cl, 5-MeO—Ph; (R⁶)ₙ is 2,6-di-F, 4-MeO; and Q is O. |
| 18C | R² is 3,5-di-MeO—Ph; (R⁶)ₙ is 2,6-di-F, 4-MeNH(CH₂)₃O; |
| | and Q is O. |
| 19C | R² is 2-Cl, 5-MeO—Ph; (R⁶)ₙ is 2,6-di-F, 4-MeNH(CH₂)₃O; |
| | and Q is O. |
| 20C | R² is 3,5-di-MeO—Ph; (R⁶)ₙ is 2,6-di-F, 3-MeO; and Q is O. |
| 21C | R² is 2-Cl, 5-MeO—Ph; (R⁶)ₙ is 2,6-di-F, 3-MeO; and Q is O. |
| 22C | R² is 2-Cl, 3,5-di-MeO—Ph; (R⁶)ₙ is 2-F; and Q is O. |
| 23C | R² is 2-Cl, 3,5-MeO—Ph; (R⁶)ₙ is 4-F; and Q is O. |
| 24C | R² is 2-Cl, 3,5-MeO—Ph; (R⁶)ₙ is 2,4-di-F; and Q is O. |
| 25C | R² is 2-Cl, 3,5-di-MeO—Ph; (R⁶)ₙ is 2,6-di-F; and Q is O. |
| 26C | R² is 2-Cl, 3,5-di-MeO—Ph; (R⁶)ₙ is 2,4,6-tri-F; and Q is O. |
| 27C | R² is 2-Cl, 3,5-di-MeO—Ph; (R⁶)ₙ is 4-Cl; and Q is O. |
| 28C | R² is 2-Cl, 3,5-di-MeO—Ph; (R⁶)ₙ is 4-MeO; and Q is O. |
| 29C | R² is 2-Cl, 3,5-di-MeO—Ph; (R⁶)ₙ is 2,3,6-tri-F; and Q is O. |
| 30C | R² is 2-Cl, 3,5-di-MeO—Ph; (R⁶)ₙ is 2,6-di-F, 4-MeO; |
| | and Q is O. |
| 31C | R² is 2-Cl, 3,5-di-MeO—Ph; (R⁶)ₙ is 2,6-di-F, |
| | 4-MeNH(CH₂)₃O; and Q is O. |
| 32C | R² is 2-Cl, 3,5-di-MeO—Ph; (R⁶)ₙ is 2,6-di-F, 3-MeO; |
| | and Q is O. |
| 33C | R² is 3,5-di-MeO—Ph; (R⁶)ₙ is 2-Cl, 6-F; and Q is O. |
| 34C | R² is 2-Cl, 5-MeO—Ph; (R⁶)ₙ is 2-Cl, 6-F; and Q is O. |
| 35C | R² is 2-Cl, 3-MeO—Ph; (R⁶)ₙ is 2-F; and Q is O. |
| 36C | R² is 2-Cl, 3-MeO—Ph; (R⁶)ₙ is 4-F; and Q is O. |
| 37C | R² is 2-Cl, 3-MeO—Ph; (R⁶)ₙ is 2,4-di-F; and Q is O. |
| 38C | R² is 2-Cl, 3-MeO—Ph; (R⁶)ₙ is 2,6-di-F; and Q is O. |
| 39C | R² is 2-Cl, 3-MeO—Ph; (R⁶)ₙ is 2,4,6-tri-F; and Q is O. |
| 40C | R² is 2-Cl, 3-MeO—Ph; (R⁶)ₙ is 2,3,6-tri-F; and Q is O. |
| 41C | R² is 2-Cl, 3-MeO—Ph; (R⁶)ₙ is 2,6-di-F, 4-OMe; and Q is O. |
| 42C | R² is 2-Cl, 3-MeO—Ph; (R⁶)ₙ is 2-Cl,6-F; and Q is O. |
| 43C | R² is 2-Cl, 3-MeO—Ph; (R⁶)ₙ is 2-Cl,4-F; and Q is O. |
| 44C | R² is 2-Cl, 3-MeO—Ph; (R⁶)ₙ is 2-F; and Q is O. |
| 45C | R² is 2-Cl—Ph; (R⁶)ₙ is 4-F; and Q is O. |
| 46C | R² is 2-Cl—Ph; (R⁶)ₙ is 2,4-di-F; and Q is O. |
| 47C | R² is 2-Cl—Ph; (R⁶)ₙ is 2,6-di-F; and Q is O. |
| 48C | R² is 2-Cl—Ph; (R⁶)ₙ is 2,4,6-tri-F; and Q is O. |
| 49C | R² is 2-Cl—Ph; (R⁶)ₙ is 2,3,6-tri-F; and Q is O. |
| 50C | R² is 2-Cl—Ph; (R⁶)ₙ is 2,6-di-F, 4-OMe; and Q is O. |
| 51C | R² is 2-Cl—Ph; (R⁶)ₙ is 2-Cl,6-F; and Q is O. |
| 52C | R² is 2-Cl—Ph; (R⁶)ₙ is 2-Cl,4-F; and Q is O. |
| 53C | R² is 3,5-di-MeO—Ph; (R⁶)ₙ is 2,4-di-F; and Q is S. |
| 54C | R² is 2-Cl, 5-MeO—Ph; (R⁶)ₙ is 2,4-di-F; and Q is S. |
| 55C | R² is 3,5-di-MeO—Ph; (R⁶)ₙ is 2,6-di-F; and Q is S. |
| 56C | R² is 2-Cl, 5-MeO—Ph; (R⁶)ₙ is 2,6-di-F; and Q is S. |
| 57C | R² is 3,5-di-MeO—Ph; (R⁶)ₙ is 2,4,6-tri-F; and Q is S. |
| 58C | R² is 2-Cl, 5-MeO—Ph; (R⁶)ₙ is 2,4,6-tri-F; and Q is S. |

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

Weight Percent

| | Active | | |
|---|---|---|---|
| | Ingredient | Diluent | Surfactant |
| Water-Dispersible and | 0.001-90 | 0-99.999 | 0-15 |
| Water-soluble Granules, | | | |
| Tablets and Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food—Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

### High Strength Concentrate

| | |
|---|---|
| Compound No. 58 | 98.5% |
| silica aerogel | 0.5% |
| synthetic amorphous fine silica | 1.0% |

### Example B

### Wettable Powder

| | |
|---|---|
| Compound No. 37 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

### Granule

| | |
|---|---|
| Compound No. 55 | 10.0% |
| attapulgite granules (low volatile matter, 0.71/0.30 mm; | 90.0% |
| U.S.S. No. 25-50 sieves) | |

### Example D

### Extruded Pellet

| | |
|---|---|
| Compound No. 54 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

### Emulsifiable Concentrate

| | |
|---|---|
| Mixture No. 57 | 10.0% |
| polyoxyethylene sorbitol hexoleate | 20.0% |
| C₆-C₁₀ fatty acid methyl ester | 70.0% |

### Example F

### Microemulsion

| | |
|---|---|
| Compound No. 58 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

### Seed Treatment

| | |
|---|---|
| Compound No. 54 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→4⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, N-[2-(1S,2R)-[1,1′-bicyclopropyl]-2-ylphenyl]-3-(difluoromethyl)-1-methyl-1H-pyrazole-4-carboxamide and penflufen (N-[2-(1,3-dimethyl-butyl)phenyl]-5-fluoro-1,3-dimethyl-1H-pyrazole-4-carboxamide). The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071), picoxystrobin and pyraoxystrobin (SYP-3343). The methoxycarbamates include pyraclostrobin and pyrametostrobin (SYP-4155). The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-prop en-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen and tebufloquin are examples of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code

20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb, valifenalate and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-5-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7), (46.4) “benzophenone fungicides” (Fungicide Resistance Action Committee (FRAC) code U8) and (46.5) “triazolopyrimidine fungicides”. The thiazole carboxamides include ethaboxam. The phenyl-acetamides include cyflufenamid and N-[[(cyclopropylmethoxy)-amino][6-(difluoromethoxy)-2,3-difluorophenyl]-methylene]benzeneacetamide. The quinazolinones include proquinazid. The benzophenones include metrafenone. The triazolopyrimidines include ametoctradin. The (b46) class also includes bethoxazin, fluxapyroxad, neo-asozin (ferric methanearsonate), pyriofenone, pyrrolnitrin, quinomethionate, tebufloquin, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethylsulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl)[1,2,4]triazolo[1,5-c]pyrimidine, N-(4-chloro-2-nitrophenyl)-N-ethyl-4-methylbenzenesulfonamide, N-[[(cyclopropylmethoxy)-amino][6-(difluoromethoxy)-2,3-difluorophenyl]methylene]benzeneacetamide, N′-[4-[4-chloro-3-(trifluoromethyl)phenoxy]-2,5-dimethylphenyl]-N-ethyl-N-methylmethanimidamide, 1-[(2-prop enylthio)carbonyl]-2-(1-methylethyl)-4-(2-methylphenyl)-5-amino-1H-pyrazol-3-one, N-[9-(dichloromethylene)-1,2,3,4-tetrahydro-1,4-methanonaphthalen-5-yl]-3-(difluoromethyl)-1-methyl-1H-pyrazole-4-carboxamide, 3-(difluoromethyl)-N-[9-(difluoro-methylene)-1,2,3,4-tetrahydro-1,4-methanonaphthalen-5-yl]-1-methyl-1H-pyrazole-4-carboxamide, N-[9-(dibromomethylene)-1,2,3,4-tetrahydro-1,4-methanonaphthalen-5-yl]-3-(difluoromethyl)-1-methyl-1H-pyrazole-4-carboxamide, N-[9-(dibromomethylene)-1,2,3,4-tetrahydro-1,4-methanonaphthalen-5-yl]-1-methyl-3-(trifluoromethyl)-1H-pyrazole-4-carboxamide, N-[9-(difluoromethylene)-1,2,3,4-tetrahydro-1,4-methanonaphthalen-5-yl]-1-methyl-3-(trifluoromethyl)-1H-pyrazole-4-carboxamide and N-[9-(dichloromethylene)-1,2,3,4-tetrahydro-1,4-methanonaphthalen-5-yl]-1-methyl-3-(trifluoromethyl)-1H-pyrazole-4-c arboxamide.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acrinathrin, amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, meperfluthrin, metaflumizone, metaldehyde, methamidophos, methidathion, methomyl, methoprene, methoxychlor, methoxyfenozide, metofluthrin, milbemycin oxime, monocrotophos, nicotine, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) proquinazid (6-iodo-3-propyl-2-propyloxy-4(3H)-quinazolinone); (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) prop amo carb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

The following Tests demonstrate the control efficacy of compounds of this invention on specific pathogens. The pathogen control protection afforded by the compounds is not limited, however, to these species. See Index Table A for compound descriptions. See Index Table B for ¹H NMR data. The following abbreviations are used in the Index Table which followi is iso, Me is methyl, Pr is propyl, i-Pr is isopropyl, Ph is phenyl, MeO is methoxy. The abbreviation “Cmpd.” stands for “Compound”, and the abbreviation “Ex.” stands for “Example” and is followed by a number indicating in which example the compound is prepared. In Index Table A the numerical value reported in the column “AP⁺(M+1)”, is the molecular weight of the observed molecular ion formed by addition of H⁺ (molecular weight of 1) to the molecule having the greatest isotopic abundance (i.e. M). The presence of molecular ions containing one or more higher atomic weight isotopes of lower abundance (e.g., ³⁷C, ⁸¹C) is not reported. The reported M+1 peaks were observed by mass spectrometry using atmospheric pressure chemical ionization (AP⁺).

INDEX TABLE A

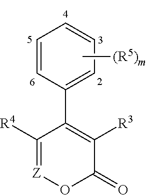

| Cmpd | | | | | | AP⁺ |
|---|---|---|---|---|---|---|
| No. | Z | R¹ | R⁴ | (R⁵) | R³ | (M + 1) |
| 1 | N | — | Me | — | 3-F—Ph | * |
| 2 | N | — | Me | 2,6-di-F | 4-Cl—Ph | * |
| 3 | N | — | Me | 4-Cl | 2,4,6-tri-F—Ph | * |
| 4 | N | — | Me | 2,6-di-F | 3-F—Ph | * |
| 5 | N | — | Me | 3-F | 2,4,6-tri-F—Ph | * |
| 6 | N | — | Me | 4-Cl | 2,3,6-tri-F—Ph | * |
| 7 | N | — | Me | 4-Cl | 2,6-diF—Ph | * |
| 8 | N | — | Me | 4-Cl | 2-Cl,6-F—Ph | * |
| 9 | N | — | Me | 4-Me | 2,4,6-tri-F—Ph | * |
| 10 | N | — | Me | 4-Br | 2,4,6-tri-F—Ph | * |
| 11 | N | — | Me | 4-F | 2,4,6-tri-F—Ph | * |
| 12 | N | — | Me | 4-CF₃ | 2,4,6-tri-F—Ph | * |
| 13 | N | — | Me | 4-Cl | 2,4,di-F—Ph | * |
| 14 | N | — | Me | 4-Br | 2-Cl-4-F—Ph | * |
| 15 | N | — | Me | 4-Br | 2,6-di-F—Ph | * |
| 16 | N | — | Me | 4-Cl, 3-F | 2,4,6-tri-F—Ph | * |
| 17 | N | — | Ph | 3,5-di-MeO | 2-F—Ph | 404 |
| [Note 1] | | | | | | |
| 17 | N | — | 3,5-di-MeO | Ph | 2-F—Ph | 404 |
| [Note 1] | | | | | | |
| 18 | N | — | Ph | 3,5-di-MeO | 2,4-di-F—Ph | 422 |
| [Note 2] | | | | | | |
| 18 | N | — | 3,5-di-MeO | Ph | 2,4-di-F—Ph | 422 |
| [Note 2] | | | | | | |
| 19 | N | — | Ph | 3,5-di-MeO | 2,6-di-F—Ph | 422 |
| [Note 3] | | | | | | |
| 19 | N | — | 3,5-di-MeO | Ph | 2,6-di-F—Ph | 422 |
| [Note 3] | | | | | | |
| 20 | N | — | 2-F—Ph | 3,5-di-MeO | 2-F—Ph | 418 |
| | | | | | | [M − 1] |
| 21 | N | — | 2-F—Ph | 3,5-di-MeO | 2,4-di-F—Ph | 438 |
| 22 | N | — | 2-F—Ph | 3,5-di-MeO | 2,6-di-F—Ph | 440 |
| 23 | N | — | 2-F—Ph | 3,5-di-MeO | 2,4,6-tri-F—Ph | * |
| (Ex. 1) | | | | | | |
| 24 | N | — | Me | 3,5-di-MeO | 2,4,6-tri-F—Ph | * |
| 25 | N | — | Me | 2-Cl-3,5-di-MeO | 2,4,6-tri-F—Ph | * |
| 26 | C | Me | 2-F—Ph | 3,5-di-MeO | Ph | 417 |
| 27 | C | Me | 2,4-di-F—Ph | 3,5-di-MeO | Ph | 435 |
| 28 | C | Me | 2-F—Ph | 3-MeO | Ph | 387 |
| 29 | C | Me | 2,4-di-F—Ph | 3-MeO | Ph | 405 |
| 30 | C | Me | 2-F—Ph | 2-Cl | Ph | 391 |
| 31 | C | Me | 2,4-di-F—Ph | 2-Cl | Ph | 409 |
| 32 | C | Me | 2-Cl-4-F—Ph | 3,5-di-MeO | Me | 389 |
| [Note 4] | | | | | | |
| 32 | C | Me | 3,5-di-MeO | 2-Cl-4-F—Ph | Me | 389 |
| [Note 4] | | | | | | |
| 33 | C | Me | 2,4-di-F—Ph | 3,5-di-MeO | 2-F—Ph | 453 |
| 34 | C | Me | 2,4-di-F—Ph | 3-MeO | 2-F—Ph | 423 |
| 35 | C | Me | 2,4-di-F—Ph | 2-Cl | 2-F—Ph | 427 |
| 36 | C | Me | 2,4-di-F—Ph | 3,5-di-MeO | Me | 370 |
| [Note 5] | | | | | | |
| 36 | C | Me | 3,5-di-MeO | 2,4-di-F—Ph | Me | 370 |
| [Note 5] | | | | | | 493 |
| 37 | C | Me | 2,4,6-tri-F—Ph | 3,5-di-MeO | 2-F—Ph | [M + Na] |
| 37 | C | Me | 2,6-di-F-4-MeO—Ph | 3,5-di-MeO | Me | 403 |
| [Note 6] | | | | | | |
| 37 | C | Me | 3,5-di-MeO | 2,6-di-F-4-MeO—Ph | Me | 403 |
| [Note 6] | | | | | | |
| 39 | C | Me | 2,6-di-F-4-MeO—Ph | 3,5-di-MeO | 2-F—Ph | 483 |
| 40 | C | Me | 2,6-di-F—Ph | 3,5-di-MeO | Ph | * |
| 41 | C | Me | Br | 2,4-di-F | Me | ** |
| (Ex. 2) | | | | | | |
| 42 | C | Me | 3,5-di-MeO—Ph | 2,4-di-F | Me | ** |
| (Ex. 3) | | | | | | |
| 43 | C | Me | 3,5-di-MeO—Ph | 2,6-di-F-4-MeO | Me | 403 |
| 44 | C | Me | 2,6-di-F-4-MeO—Ph | 2,6-di-Cl-3,5-di-MeO | Me | 473 |
| 45 | C | Me | 4-F—Ph | 3,5-di-MeO | i-Pr | 383 |
| [Note 7] | | | | | | |
| 45 | C | Me | 3,5-di-MeO | 4-F—Ph | i-Pr | 383 |
| [Note 7] | | | | | | |
| 46 | C | Me | 2-Cl-4-F—Ph | 3,5-di-MeO | Me | 389 |
| 47 | C | Me | 4-F—Ph | 3,5-di-MeO | H | 341 |
| [Note 8] | | | | | | |
| 47 | C | Me | 3,5-di-MeO | 4-F—Ph | H | 341 |
| [Note 8] | | | | | | |
| 48 | C | Me | 2,4-di-F—Ph | 3,5-di-MeO | Me | 373 |
| 49 | C | Me | 2-Cl-3,5-di-MeO—Ph | 2,6-di-F-4-MeO | Me | 437 |
| 50 | C | Me | 2-Cl-4-F—Ph | 2-Cl-3,5-di-MeO | Me | 425 |
| [Note 9] | | | | | | |
| 50 | C | Me | 2-Cl-3,5-di-MeO | 2-Cl-4-F—Ph | Me | 425 |
| [Note 9] | | | | | | |
| 51 | C | Me | 2,6-di-F—Ph | 3,5-di-MeO | Me | 373 |
| 52 | C | Me | 2,6-di-F—Ph | 2-Cl-3-MeO | Me | 377 |
| [Note 10] | | | | | | |
| 52 | C | Me | 2-Cl-3-MeO | 2,6-di-F—Ph | Me | 377 |
| [Note 10] | | | | | | |
| 53 | C | Me | 2,6-di-F—Ph | 2,6-di-Cl-3,5-di-MeO | Me | 443 |
| 54 | C | Me | 2-Cl-4-F—Ph | 2-Cl-3,5-di-MeO | Me | 425 |
| 55 | C | Me | 2,4,6-tri-F—Ph | 3,5-di-MeO | Me | 391 |
| (Ex. 4) | | | | | | |
| 56 | C | Me | 2-Cl, 4-F—Ph | 3,5-di-MeO | H | 375 |
| 57 | C | Me | 2,4,6-tri-F—Ph | 2-Cl-3,5-di-MeO | Me | 375 |
| (Ex. 5) | | | | | | |
| [Note 11] | | | | | | |
| 57 | C | Me | 2-Cl-3,5-di-MeO | 2,4,6-tri-F—Ph | Me | 375 |
| [Note 11] | | | | | | |
| 58 | C | Me | 2,4,6-tri-F—Ph | 2-Cl-3,5-di-MeO | Me | 461 |
| (Ex. 6) | | | | | | |
| 59 | C | Me | 2,4,6-tri-F—Ph | 2,6-di-Cl-3,5-di-MeO | Me | 461 |
| 60 | C | Me | 2-F—Ph | 3,5-di-MeO | Me | 355 |

*See Index Table B for ¹H NMR data.
**See synthesis example for ¹H NMR data.
[Note 1]: Mixture of regioisomers in a ratio of about 76:24 of the first compound listed in the Index Table to the
second for number 17.
[Note 2]: Mixture of regioisomers in a ratio of about 92:8 of the first compound listed in the Index Table to the
second for number 18.
[Note 3]: Mixture of regioisomers in a ratio of about 52:48 of the first compound listed in the Index Table to the
second for number 19.
[Note 4]: Mixture of regioisomers in a ratio of about 60:40 of the first compound listed in the Index Table to the
second for number 32.
[Note 5]: Mixture of regioisomers in a ratio of about 60:40 of the first compound listed in the Index Table to the
second for number 36.
[Note 6]: Mixture of regioisomers in a ratio of about 20:80 of the first compound listed in the Index Table to the
second for number 37.
[Note 7]: Mixture of regioisomers in a ratio of about 60:40 of the first compound listed in the Index Table to the
second for number 45.
[Note 8]: Mixture of regioisomers in a ratio of about 50:50 of the first compound listed in the Index Table to the
second for number 47.
[Note 9]: Mixture of regioisomers in a ratio of about 50:50 of the first compound listed in the Index Table to the
second for number 50.
[Note 10]: Mixture of regioisomers in a ratio of about 60:40 of the first compound listed in the Index Table to the
second for number 52.
[Note 11]: Mixture of regioisomers in a ratio of about 50:50 of the first compound listed in the Index Table to the
second for number 57.

INDEX TABLE B

| Compd. No. | ¹H NMR Data (CDCl₃ solution unless indicated otherwise)^{a} |
|---|---|
| 1 | δ 2.14 (s, 3H), 6.85-7.35 (m, 9H). |
| 2 | δ 2.15 (s, 3H), 6.92 (m, 2H), 7.14 (m, 2H), 7.24 (m, 2H), 7.35 (m, 1H). |
| 3 | δ 2.15 (s, 3H), 6.59(m, 2H), 7.05 (m, 2H), 7.35 (m, 2H). |
| 4 | δ 2.15 (s, 3H), 6.92 (m, 5H), 7.22 (m, 1H), 7.35 (m, 1H). |
| 5 | δ 2.16 (s, 3H), 6.59 (m, 2H), 6.93 (m, 2H), 7.06 (m, 1H), 7.34 (m, 1H). |
| 6 | δ 2.16 (s, 3H), 6.75 (m, 1H), 7.06 (m, 2H), 7.12 (m, 1H), 7.35 (m, 2H). |
| 7 | δ 2.15 (s, 3H), 6.82 (m, 2H), 7.07 (m, 2H), 7.31 (m, 3H). |
| 8 | δ 2.15 (s, 3H), 6.82 (m, 1H), 7.09 (m, 2H), 7.15 (m, 1H), 7.31 (m, 3H). |
| 9 | δ 2.15 (s, 3H), 2.24 (s, 3H), 6.55 (m, 2H), 6.98 (m, 2H), 7.13 (m, 2H). |
| 10 | δ 2.15 (s, 3H), 6.60 (m, 2H), 6.98 (m, 2H), 7.50 (m, 2H). |
| 11 | δ 2.15 (s, 3H), 6.59 (m, 2H), 7.08 (m, 4H). |
| 12 | δ 2.15 (s, 3H), 6.59 (m, 2H), 7.25 (m, 2H), 7.65 (m, 2H). |
| 13 | δ 2.14 (s, 3H), 6.74 (m, 1H), 6.80 (m, 1H), 7.06 (m, 3H), 7.33 (m, 2H). |
| 14 | δ 2.14 (s, 3H), 6.88 (m, 1H), 6.93 (m, 1H), 6.98 (m, 2H), 7.11 (m, 1H), |
| | 7.46 (m, 2H). |
| 15 | δ 2.15 (s, 3H), 6.83 (m, 2H), 6.95 (m, 2H), 7.31 (m, 1H), 7.47 (m, 2H). |
| 16 | δ 2.16 (s, 3H), 6.64 (m, 2H), 6.85 (m, 1H), 6.93 (m, 1H), 7.41 (m, 1H). |
| 23 | δ 3.52 (s, 6H), 6.00 (m, 2H), 6.25 (m, 1H), 6.65 (m, 1H), 6.92 (m, 2H), |
| | 7.17 (m, 1H), 7.36 (m, 2H). |
| 24 | δ 2.16 (s, 3H), 3.72 (s, 6H), 6.21 (m, 2H), 6.41 (m, 1H), 6.60 (m, 2H). |
| 25 | δ 2.15 (s, 3H), 3.73 (s, 3H), 3.86 (s, 3H), 6.22 (m, 1H), 6.48 (m, 1H), |
| | 6.60 (m, 2H). |
| 40 | δ 2.18 (s, 3H), 3.48 (s, 6H), 5.97 (m, 2H), 6.08 (m, 2H), 6.82 (m, 2H), |
| | 7.16 (m, 5H). |
| 41 | δ 1.91 (s, 3H), 2.46 (s, 3H), 6.95 (m, 1H), 7.00 (m, 1H), 7.09 (m, 1H). |
| 42 | δ 1.89 (s, 3H), 2,15 (s, 3H), 3.65 (s, 3H), 3.69 (s, 3H), 6.22 (m, 2H), |
| | 6.26 (m, 1H), 6.73 (m, 2H), 6.84 (m, 1H). |

^{a1}H NMR data are in ppm downfield from tetramethylsilane in CDCl3 solution at 400 MHz. Couplings are designated by (s)—singlet and (m)—multiplet.

### Biological Examples of the Invention

General protocol for preparing test suspensions for Tests A-I: the test compounds were first dissolved in acetone in an amount equal to 3% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-I. Spraying a 200 ppm test suspension to the point of run-off on the test plants was the equivalent of a rate of 500 g/ha. Unless otherwise indicated, the rating values indicate a 200 ppm test suspension was used. (An asterisk “*” next to the rating value indicates a 40 ppm test suspension was used.)

### Test A

The test suspension was sprayed to the point of run-off on tomato seedlings. The following day, the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 additional days, after which time visual disease ratings were made.

### Test B

The test suspension was sprayed to the point of run-off on tomato seedlings. The following day, the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time visual disease ratings were made.

### Test C

The test suspension was sprayed to the point of run-off on tomato seedlings. The following day, the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which time visual disease ratings were made.

### Test D

The test suspension was sprayed to the point of run-off on creeping bent grass (Agrostis sp.) seedlings. The following day, the seedlings were inoculated with a bran and mycelial slurry of Rhizoctonia solani (the causal agent of turf brown patch) and incubated in a saturated atmosphere at 27° C. for 48 h, and then moved to a growth chamber at 27° C. for 3 days, after which time disease ratings were made.

### Test E

The test suspension was sprayed to the point of run-off on wheat seedlings. The following day the seedlings were inoculated with a spore suspension of Septoria nodorum (the causal agent of Septoria glume blotch) and incubated in a saturated atmosphere at 24° C. for 48 h, and then moved to a growth chamber at 20° C. for 9 days, after which time visual disease ratings were made.

### Test F

The test suspension was sprayed to the point of run-off on wheat seedlings. The following day, the seedlings were inoculated with a spore suspension of Septoria tritici (the causal agent of wheat leaf blotch) and incubated in a saturated atmosphere at 24° C. for 48 h, and then the seedlings were moved to a growth chamber at 20° C. for 19 additional days, after which time visual disease ratings were made.

### Test G

Wheat seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 2 days. At the end of this time, the test suspension was sprayed to the point of run-off, and then the seedlings were moved to a growth chamber at 20° C. for 4 days after which time visual disease ratings were made.

### Test H

The test suspension was sprayed to the point of run-off on wheat seedlings. The following day, the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 6 days, after which time visual disease ratings were made

### Test I

The test suspension was sprayed to the point of run-off on wheat seedlings. The following day, the seedlings were inoculated with a spore dust of Erysiphe graminis f. sp. tritici, (the causal agent of wheat powdery mildew) and incubated in a growth chamber at 20° C. for 7 days, after which time visual disease ratings were made.

Results for Tests A-I are given in Table A. In the table, a rating of 100 indicates 100% disease control and a rating of 0 indicates no disease control (relative to the controls). A dash (-) indicates no test results. All results are for 200 ppm except where followed by an asterisk “*”, which indicates a 40 ppm.

| No. | Test A | Test B | Test C | Test D | Test E | Test F | Test G | Test H | Test I |
|---|---|---|---|---|---|---|---|---|---|
| 1 | 0 | — | 0 | 0 | 0 | 37 | — | 0 | 0 |
| 2 | 47 | — | 17 | 0 | 0 | 97 | — | 0 | 92 |
| 3 | 99 | 100 | 9 | 78 | 0 | 99 | — | 98 | 99 |
| 4 | 0 | | 8 | 0 | 0 | 0 | — | 0 | 0 |
| 5 | 0 | 54 | 0 | 16 | 0 | — | — | 0 | 20 |
| 6 | 99 | 99 | 0 | 0 | 0 | — | — | 41 | 96 |
| 7 | 100 | 100 | 0 | 75 | 0 | — | — | 99 | 100 |
| 8 | 100 | 99 | 0 | 52 | 0 | — | — | 98 | 99 |
| 9 | 90 | 46 | 0 | 99 | 0 | — | — | 96 | 96 |
| 10 | 98 | 100 | 0 | 90 | 0 | — | — | 95 | 99 |
| 11 | 100 | 99 | 0 | 26 | 64 | — | — | 98 | 100 |
| 12 | 0 | 99* | 0 | 0 | 0 | — | — | 0 | 98 |
| 13 | 0 | 9 | 0 | 0 | 0 | — | — | 91 | 99 |
| 14 | 0 | 5 | 0 | 0 | 0 | — | — | 0 | 0 |
| 15 | 0 | 96 | 0 | 0 | 0 | — | — | 91 | 98 |
| 16 | 0 | 0 | 0 | 0 | 0 | 97 | — | 0 | 0 |
| 17 | 34 | 0 | 0 | 0 | 0 | 0 | — | 74 | 0 |
| 18 | 99 | 0 | 0 | 0 | 0 | 93 | — | 80 | 76 |
| 19 | 99 | 0 | 0 | 9 | 0 | 34 | — | 95 | 64 |
| 20 | 0 | 0 | 0 | 0 | 0 | 0 | — | 0 | 0 |
| 21 | 0 | 0 | 0 | 0 | 0 | 0 | — | 0 | 0 |
| 22 | 0 | 0 | 0 | 8 | 0 | 0 | — | 0 | 0 |
| 23 | 100 | 93 | — | — | 95 | 99 | 9 | 100 | 0 |
| 24 | 98 | 63 | — | — | 0 | 99 | 0 | | 99 |
| 25 | 100 | 97 | — | — | 0 | 100 | 100 | 100 | 100 |
| 26 | 99 | 93 | — | — | 0 | 95 | 9 | 100 | 27 |
| 27 | 98 | 100 | — | — | 99 | 99 | 93 | 100 | 82 |
| 28 | 100 | 0 | — | — | 0 | 98 | 0 | 99 | 64 |
| 29 | 99 | 93 | — | — | 64 | 99 | 32 | 100 | 55 |
| 30 | 99* | 0* | — | — | 0* | 94* | 0* | 91* | 0* |
| 31 | 100* | 0* | — | — | 0* | 98* | 96* | 99* | 0 |
| 32 | 99 | 99 | 0 | — | 90 | 100 | — | 100 | 99 |
| 33 | 99 | 100 | — | — | 99* | 98 | 99 | 100 | 76 |
| 34 | 100 | 99 | — | — | 99 | 99 | 95 | 100 | 90 |
| 35 | 100* | 68* | — | — | 0* | 99* | 99* | 0* | 97 |
| 36 | 99 | 99 | 0 | 99 | 99 | 100 | 74 | 99 | 99 |
| 37 | 93* | 100* | — | — | 99* | 100* | 100* | 100* | 100 |
| 38 | 100 | 100 | — | — | 99 | 100 | 99 | 100 | 100 |
| 39 | 100* | 100* | — | — | 100* | 100* | 82* | 100* | 79 |
| 40 | 100* | 100* | — | — | 90* | 100* | 95* | 100* | 89 |
| 41 | 67 | 0 | — | — | 0 | 0 | 0 | 0 | 0 |
| 42 | 98 | 0 | — | — | 0 | 100 | 0 | 100 | 99 |
| 43 | 100 | 100 | — | — | 100 | 100 | 98 | 100 | 100 |
| 44 | 79 | 0 | — | — | 0 | 73 | 26 | 96 | 0 |
| 45 | 99 | 0 | — | — | 0 | 100 | 0 | 74 | 0 |
| 46 | 100 | 98 | — | — | 95 | 100 | 79 | 100 | 99 |
| 47 | 100 | 93 | — | — | 94 | 100 | 9 | 100 | 64 |
| 48 | 100 | 97 | — | — | 99 | 100 | 100 | 100 | 100 |
| 49 | 100 | 100 | — | — | 99 | 100 | 100 | 100 | 99 |
| 50 | 100 | 99 | — | — | 98 | 100 | 100 | 100 | 100 |
| 51 | 100 | 0 | — | — | 0 | 100 | 75 | 100 | 96 |
| 52 | 100 | 26 | — | — | 0 | 100 | 100 | 100 | 100 |
| 53 | 61 | 0 | — | — | 0 | 47 | 0 | 88 | 0 |
| 54 | 100 | 96 | — | — | 100 | 100 | 99 | 100 | 100 |
| 55 | 77 | 100 | — | — | 99 | 100 | 100 | 100 | 100 |
| 56 | 94 | 99 | — | — | 99 | 100 | 100 | 100 | 100 |
| 57 | 100 | 100 | — | — | 100 | 100* | 100 | 100 | 100 |
| 58 | 100 | 100 | — | — | 100 | 100* | 100 | 100 | 100 |
| 59 | 100 | 0 | — | — | 0 | — | 82 | 100 | 90 |
| 60 | — | 26 | — | — | 0 | 100 | 96 | 100 | 98 |

What is claimed is:

1. A compound selected from Formula 1, N-oxides and salts thereof, 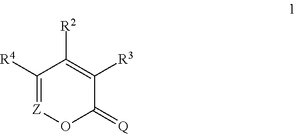
wherein
Q is O or S;
Z is N or CR¹;
R¹ is H, halogen, cyano, C₁-C₄ alkyl, C₂-C₄ alkenyl, C₂-C₄ alkynyl, C₁-C₄ haloalkyl, C₂-C₄ haloalkenyl, C₂-C₄ haloalkynyl, cyclopropyl, halocyclopropyl, C₂-C₄ alkoxyalkyl, C₂-C₄ alkylthioalkyl, C₂-C₄ alkylsulfinylalkyl, C₂-C₄ alkylsulfonylalkyl, C₂-C₄ cyanoalkyl, C₁-C₃ hydroxyalkyl, C₁-C₃ alkoxy, C₁-C₃ haloalkoxy, C₁-C₃ alkylthio, C₁-C₃ haloalkylthio, C₁-C₃ alkylamino or C₂-C₄ dialkylamino;
R² is a phenyl ring substituted with up to 5 substituents independently selected from R⁵; or a 3- to 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from R⁵ on carbon atom ring members and R^{5a} on nitrogen atom ring members;
R³ is a phenyl ring optionally substituted with up to 5 substituents independently selected from R⁶; or a 3- to 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from R⁶ on carbon atom ring members and R^{6a} on nitrogen atom ring members; or H, halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₃-C₆ cycloalkylamino, C₂-C₆ alkylcarbonylamino, C₂-C₆ haloalkylcarbonylamino, C₁-C₆ alkylsulfonylamino or C₁-C₆ halo alkylsulfonylamino;
R⁴ is a phenyl ring optionally substituted with up to 5 substituents independently selected from R⁷; or a 3- to 6-membered heterocyclic ring containing ring members selected from carbon atoms and up to 4 heteroatoms independently selected from up to 2 O, up to 2 S and up to 3 N atoms, wherein up to 3 carbon atom ring members are independently selected from C(═O) and C(═S), and the sulfur atom ring members are independently selected from S(═O)ₚ(═NR⁸)_{q}, the ring optionally substituted with up to 5 substituents independently selected from R⁷ on carbon atom ring members and R^{7a} on nitrogen atom ring members; or halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₆-C₁₂ cycloalkylcycloalkyl, C₄-C₈ halocycloalkylalkyl, C₅-C₈ alkylcycloalkylalkyl, C₃-C₆ cycloalkenyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₂-C₆ alkylaminocarbonyl, C₃-C₈ dialkylaminocarbonyl, C₂-C₆ cyanoalkyl, C₁-C₆ hydroxyalkyl, C₂-C₆ hydroxyhaloalkyl, C₂-C₆ hydroxyalkylcarbonyl, C₂-C₆ hydroxycarbonylalkyl, C₁-C₆ alkoxy, C₁-C₆ haloalkoxy, C₃-C₆ cycloalkoxy, C₃-C₆ halocycloalkoxy, C₂-C₆ alkoxyalkoxy, C₃-C₆ alkoxycarbonylalkyl,
C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₁-C₆ alkylamino, C₂-C₆ dialkylamino, C₂-C₆ haloalkylamino, C₂-C₆ halodialkylamino, C₃-C₆ cycloalkylamino, C₂-C₆ alkylcarbonylamino, C₂-C₆ haloalkylcarbonylamino, C₁-C₆ alkylsulfonylamino or C₁-C₆ haloalkylsulfonylamino;
each R⁵ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino; or
a pair of R⁵ attached to the same carbon atom are taken together to form a 5- to 7-membered spirocyclic ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro, C₁-C₂ alkyl and C₁-C₂ alkoxy on carbon atom ring members and cyano, C₁-C₂ alkyl and C₁-C₂ alkoxy on nitrogen atom ring members;
each R^{5a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl; or
a pair of R⁵ and R^{5a} attached to adjacent ring atoms are taken together to form a 5- to 7-membered ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl, and C₁-C₂ alkoxy on nitrogen atom ring members;
each R⁶ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino; or
a pair of R⁶ attached to the same carbon atom are taken together to form a 5- to 7-membered spirocyclic ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl and C₁-C₂ alkoxy on nitrogen atom ring members;
each R^{6a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl; or
a pair of R⁶ and R^{6a} attached to adjacent ring atoms are taken together to form a 5- to 7-membered ring containing ring members selected from carbon atoms and optionally up to 4 heteroatoms independently selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl and C₁-C₂ alkoxy on nitrogen atom ring members;
each R⁷ is independently halogen, cyano, hydroxy, amino, nitro, —CHO, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ alkylaminoalkoxy, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₂-C₆ cyanoalkyl, C₁-C₆ hydroxyalkyl, C₁-C₆ alkoxy, C₁-C₆ haloalkoxy, C₃-C₆ cycloalkoxy, C₃-C₆ halocycloalkoxy, C₂-C₆ alkylcarbonyloxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₃-C₉ trialkylsilyl, C₂-C₆ alkylcarbonylthio, C₁-C₆ alkylamino or C₂-C₆ dialkylamino; or
a pair of R⁷ attached to adjacent carbon atoms are taken together to form a 5- to 7-membered ring containing ring members selected from carbon atoms and optionally up to 4 heteroatoms selected from up to 2 O, up to 2 S and up to 3 N atoms, the ring optionally substituted with up to 3 substituents independently selected from halogen, cyano, nitro C₁-C₂ alkyl and C₁-C₂ alkoxy on carbon atom ring members and cyano C₁-C₂ alkyl and C₁-C₂ alkoxy on nitrogen atom ring members;
each R^{7a} is independently cyano, C₁-C₆ alkyl, C₂-C₆ alkenyl, C₂-C₆ alkynyl, C₁-C₆ haloalkyl, C₂-C₆ alkylcarbonyl, C₂-C₆ haloalkylcarbonyl, C₂-C₆ alkoxycarbonyl, C₂-C₆ alkylaminocarbonyl, C₃-C₆ dialkylaminocarbonyl, C₂-C₆ haloalkenyl, C₂-C₆ haloalkynyl, C₃-C₆ cycloalkyl, C₃-C₆ halocycloalkyl, C₄-C₈ alkylcycloalkyl, C₄-C₈ cycloalkylalkyl, C₅-C₈ alkylcycloalkylalkyl, C₂-C₆ alkoxyalkyl, C₁-C₆ alkoxy, C₁-C₆ haloalkoxy, C₃-C₆ cycloalkoxy, C₃-C₆ halocycloalkoxy, C₁-C₆ alkylthio, C₁-C₆ haloalkylthio, C₁-C₆ alkylsulfonyl, C₁-C₆ haloalkylsulfonyl or C₃-C₉ trialkylsilyl;
each R⁸ is independently H or C₁-C₃ alkyl; and
p and q are independently 0, 1 or 2 in each instance of S(═O)ₚ(═NR⁸)_{q}, provided that the sum of p and q is 0, 1 or 2;
provided that the compound is other than 4-(4-chlorophenyl)-3,5-dimethyl-6H-1,2-oxazin-6-one,
4-[4-(dimethylamino)phenyl]-3-phenyl-6H-1,2-oxazin-6-one,
3,5-dimethyl-4-(3-pyridinyl)-6H-1,2-oxazin-6-one,
4-(4-methoxyphenyl)-3-phenyl-6H-1,2-oxazin-6-one,
3,4-diphenyl-6H-1,2-oxazin-6-one,
4,5-diphenyl-2H-pyran-2-one, or
5,6-dimethyl-3,4-diphenyl-2H-pyran-2-one.

2. A compound of claim 1 wherein:
Q is O;
R¹ is C₁-C₄ alkyl, C₂-C₄ alkenyl, C₁-C₄ haloalkyl or C₁-C₃ alkoxy;
R² is a phenyl or pyridinyl ring optionally substituted with up to 3 substituents independently selected from R⁵;
R³ is a phenyl or pyridinyl ring optionally substituted with up to 3 substituents independently selected from R⁶; or halogen or C₁-C₆ alkyl;
R⁴ is a phenyl or pyridinyl ring optionally substituted with up to 3 substituents independently selected from R⁷; or halogen or C₁-C₆ alky; and
each R⁵, R⁶ and R⁷ is independently halogen, cyano, C₁-C₆ alkyl, C₂-C₆ alkenyl, C₁-C₆ haloalkyl, C₁-C₆ alkoxy, C₁-C₆ haloalkoxy, C₁-C₆ alkylthio or C₁-C₆ haloalkylthio.

3. A compound of claim 2 wherein:
R¹ is C₁-C₄ alkyl;
R² is a phenyl ring optionally substituted with up to 3 substituents independently selected from R⁵;
R³ is a phenyl ring optionally substituted with up to 3 substituents independently selected from R⁶; or methyl or i-propyl;
R⁴ is a phenyl ring optionally substituted with up to 3 substituents independently selected from R⁷; or methyl; and
each R⁵, R⁶ and R⁷ is independently halogen, C₁-C₆ alkyl, C₂-C₆ alkenyl, C₁-C₆ haloalkyl or C₁-C₆ alkoxy.

4. A compound of claim 3 wherein:
R² is a phenyl ring substituted with 2 or 3 substituents independently selected from R⁵;
R³ is a phenyl ring substituted with 1, 2 or 3 substituents independently selected from R⁶; or methyl;
R⁴ is a phenyl ring substituted with 1, 2 or 3 substituents independently selected from R⁷; or methyl; and
each R⁵, R⁶ and R⁷ is independently halogen, methyl or methoxy.

5. A compound of claim 4 wherein:
Z is CR¹;
R¹ is methyl;
R⁴ is a phenyl ring substituted with 2 or 3 substituents independently selected from R⁷; and
each R⁵, R⁶ and R⁷ is independently chloro, fluoro or methoxy.

6. A compound of claim 5 wherein:
Z is N;
R³ is a phenyl ring substituted with 1, 2 or 3 substituents independently selected from R⁶;
R⁴ is methyl; and
each R⁵, R⁶ and R⁷ is independently chloro, fluoro or methoxy.

7. A compound of claim 1 selected from the group consisting of:
4-(2-chloro-3,5-dimethoxyphenyl)-5-(2-chloro-4-fluorophenyl)-3,6-dimethyl-2H-pyran-2-one;
5-(2,4-difluorophenyl)-4-(3,5-dimethoxyphenyl)-3,6-dimethyl-2H-pyran-2-one;
5-(2,4-difluorophenyl)-4-(3,5-dimethoxyphenyl)-3-(2-fluorophenyl)-6-methyl-2H-pyran-2-one;
4-(3,5-dimethoxyphenyl)-3-(2-fluorophenyl)-5-(2,4,6-trifluorophenyl)-6H-1,2-oxazin-6-one; and
4-(2-chloro-3,5-dimethoxyphenyl)-3-methyl-5-(2,4,6-trifluorophenyl)-6H-1,2-oxazin-6-one.

8. A fungicidal composition comprising (a) a compound of claim 1; and (b) at least one other fungicide.

9. A fungicidal composition comprising (a) a compound of claim 1; and at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.
